(19) United States
(12) Patent Application Publication
(10) Pub. No.: US 2026/0157705 A1
(43) Pub. Date: Jun. 11, 2026

(54) ANALYTE MONITORING SYSTEM INCLUDING DYNAMIC STREAM SELECTION

(71) Applicant: Senseonics, Incorporated, Germantown, MD (US)

(72) Inventors: Arnaud Jacquin, Germantown, MD (US); Samanwoy Ghosh-Dastidar, Germantown, MD (US); Mukul Jain, Germantown, MD (US); Patricia Sanchez, Germantown, MD (US); Yahya Hosseini, Germantown, MD (US)

(73) Assignee: Senseonics, Incorporated, Germantown, MD (US)

(21) Appl. No.: 19/181,620

(22) Filed: Apr. 17, 2025

Related U.S. Application Data
(60) Provisional application No. 63/636,254, filed on Apr. 19, 2024.

(51) Int. Cl. A61B 5/00; A61B 5/145

(52) U.S. Cl. CPC A61B 5/742; A61B 5/0022; A61B 5/14532

(57) ABSTRACT

Apparatuses, systems, and methods for calculating and selecting between multiple streams of analyte levels. The method may include using first and second methods to calculate streams of first and second analyte levels for multiple instances of time based on at least sensor data for the multiple instances of time. The method may include using the stream of first analyte levels for display and determining whether to switch from use of the stream of first analyte levels for display to use of the stream of second analyte levels for display, which may include comparing one or more of the first analyte levels to one or more of the second analyte levels. The method may include continuing to use the stream of first analyte levels for display or switching to use of the stream of second analyte levels for display depending on the determination of whether to switch.

### CROSS-REFERENCE TO RELATED APPLICATION

The present application claims the benefit of priority to U.S. Provisional Application Ser. No. 63/636,254, filed on Apr. 19, 2024, which is incorporated herein by reference in its entirety.

### BACKGROUND

### Field of Invention

The present disclosure relates to an analyte monitoring system and method. More specifically, aspects of the present disclosure relate to an analyte monitoring system that switches between streams of first and second analyte levels for display.

### Discussion of the Background

The prevalence of diabetes mellitus continues to increase in industrialized countries, and projections suggest that this figure will rise to 4.4% of the global population (366 million individuals) by the year 2030. Glycemic control is a key determinant of long-term outcomes in patients with diabetes, and poor glycemic control is associated with retinopathy, nephropathy and an increased risk of myocardial infarction, cerebrovascular accident, and peripheral vascular disease requiring limb amputation. Despite the development of new insulins and other classes of antidiabetic therapy, roughly half of all patients with diabetes do not achieve recommended target hemoglobin A1c (HbA1c) levels <7.0%.

Frequent self-monitoring of blood glucose (SMBG) is necessary to achieve tight glycemic control in patients with diabetes mellitus, particularly for those requiring insulin therapy. However, current blood (finger-stick) glucose tests are burdensome, and, even in structured clinical studies, patient adherence to the recommended frequency of SMBG decreases substantially over time. Moreover, finger-stick measurements only provide information about a single point in time and do not yield information regarding intraday fluctuations in blood glucose levels that may more closely correlate with some clinical outcomes.

Analyte monitoring systems (e.g., continuous glucose monitors (CGMs)) have been developed in an effort to overcome the limitations of finger-stick SMBG and thereby help improve patient outcomes. These systems enable increased frequency of glucose measurements and a better characterization of dynamic glucose fluctuations, including episodes of unrealized hypoglycemia. Furthermore, integration of CGMs with automated insulin pumps allows for establishment of a closed-loop “artificial pancreas” system to more closely approximate physiologic insulin delivery and to improve adherence.

Monitoring analyte measurements from a living body via wireless analyte monitoring sensor(s) may provide numerous health and research benefits. Improved analyte monitoring systems and methods are needed.

### SUMMARY

One aspect of the invention may provide a method including receiving sensor data for multiple instances of time. The sensor data may have been conveyed by an analyte sensor. The method may include using a first method to calculate a stream of first analyte levels for the multiple instances of time based on at least the sensor data for the multiple instances of time. The method may include using a second method to calculate a stream of second analyte levels for the multiple instances of time based on at least the sensor data for the multiple instances of time. The method may include using the stream of first analyte levels for display. The method may include determining whether to switch from use of the stream of first analyte levels for display to use of the stream of second analyte levels for display. Determining whether to switch from use of the stream of first analyte levels for display to use of the stream of second analyte levels may include comparing one or more of the first analyte levels to one or more of the second analyte levels. The method may include continuing to use the stream of first analyte levels for display if determined to not switch from use of the stream of first analyte levels to use of the stream of second analyte levels. The method may include switching to use of the stream of second analyte levels for display if determined to switch from use of the stream of first analyte levels to use of the stream of second analyte levels.

In some aspects, the first method may be one of a ratio method and a two-parameter method, and the second method may be another of the ratio method and the two-parameter method. In some aspects, using the stream of first analyte levels for display may include displaying one or more of the first analyte levels. In some aspects, using the stream of first analyte levels for display may include conveying one or more of the first analyte levels to a display device for display by the display device.

In some aspects, the method may include receiving a first reference analyte value for a first reference instance of time and receiving a second reference analyte value for a second reference instance of time, and determining whether to switch from use of the stream of first analyte levels for display to use of the stream of second analyte levels for display may be performed if the second reference analyte value is received. In some aspects, comparing the one or more of the first analyte levels to the one or more of the second analyte levels may include: determining an aggregate value of the first analyte levels that are for instances of the multiple instances of time that are between the first and second reference instances of time, determining an aggregate value of the second analyte levels that are for instances of the multiple instances of time that are between the first and second reference instances of time, and determining whether the aggregate value of the second analyte levels is lower than the aggregate value of the first analyte levels. In some aspects, determining whether to switch from use of the stream of first analyte levels for display to use of the stream of second analyte levels for display may include determining to not switch from the use of the stream of first analyte levels for display to use of the stream of second analyte levels for display if the aggregate value of the second analyte levels is not determined to be lower than the aggregate value of the first analyte levels, and determining whether to switch from use of the stream of first analyte levels for display to use of the stream of second analyte levels for display may include determining to switch from the use of the stream of first analyte levels for display to use of the stream of second analyte levels for display if the aggregate value of the second analyte levels is determined to be lower than the aggregate value of the first analyte levels. In some aspects, the determined aggregate value of the first analyte levels that are for the instances of the multiple instances of time that are between the first and second reference instances of time may be a first quartile of the first analyte levels that are for the instances of the multiple instances of time that are between the first and second reference instances of time, and the determined aggregate value of the second analyte levels that are for the instances of the multiple instances of time that are between the first and second reference instances of time may be a first quartile of the second analyte levels that are for the instances of the multiple instances of time that are between the first and second reference instances of time.

In some aspects, the method may further include: receiving a third reference analyte value for a third reference instance of time, determining a second aggregate value of the first analyte levels that are for instances of the multiple instances of time that are between the second and third reference instances of time, determining a second aggregate value of the second analyte levels that are for instances of the multiple instances of time that are between the second and third reference instances of time, determining whether the second aggregate value of the second analyte levels is lower than the second aggregate value of the first analyte levels, using the stream of first analyte levels for display for one or more instances of the multiple instances of time that are subsequent to the third reference instances of time if the second aggregate value of the second analyte levels is not determined to be lower than the second aggregate value of the first analyte levels, and using the stream of second analyte levels for display for the one or more instances of the multiple instances of time that are subsequent to the third reference instances of time if the second aggregate value of the second analyte levels is determined to be lower than the second aggregate value of the first analyte levels.

In some aspects, determining whether to switch from use of the stream of first analyte levels for display to use of the stream of second analyte levels may include (a) determining whether the first analyte level for a first instance of the multiple instances of time is less than a first analyte level threshold and/or (b) determining at the first instance of the multiple instances of time whether a predicted first analyte level is less than the first analyte level threshold. In some aspects, comparing the one or more of the first analyte levels to the one or more of the second analyte levels may include determining whether the second analyte level for the first instance of the multiple instances of time is lower than the first analyte level for the first instance of the multiple instances of time. In some aspects, comparing the one or more of the first analyte levels to the one or more of the second analyte levels may further include determining whether a difference between the first and second analyte levels for the first instance of the multiple instances of time is greater than a maximum analyte level difference threshold. In some aspects, determining whether to switch from use of the stream of first analyte levels for display to use of the stream of second analyte levels may include determining whether the first analyte level for the first instance of the multiple instances of time is less than the first analyte level threshold.

In some aspects, determining whether to switch from use of the stream of first analyte levels for display to use of the of the stream of second analyte levels for display may include determining at the first instance of the multiple instances of time whether the predicted first analyte level is less than the first analyte level threshold. In some aspects, determining whether to switch from use of the stream of first analyte levels for display to use of the stream of second analyte levels for display may include determining to not switch from the use of the stream of first analyte levels for display to use of the stream of second analyte levels for display if (a) the first analyte level for the first instance of time of the multiple instances of time is less than the second analyte level for the first instance of time and/or (b) the difference between the first and second analyte levels for the first instance of time is greater than the maximum analyte level difference threshold.

In some aspects, determining whether to switch from use of the stream of first analyte levels for display to use of the stream of second analyte levels for display may include determining to switch from the use of the stream of first analyte levels for display to use of the stream of second analyte levels for display if (i) the first analyte level for the first instance of time is greater than the second analyte level for the first instance of time and (ii) the difference between the first and second analyte levels for the first instance of time is less than the analyte difference threshold.

In some aspects, determining whether to switch from use of the stream of first analyte levels for display to use of the stream of second analyte levels for display further may include determining whether a rate of change of the stream of first analyte levels at the first instance of the multiple instances of time is less than or equal to a falling rate of change threshold. In some aspects, determining whether to switch from use of the stream of first analyte levels for display to use of the stream of second analyte levels for display may include determining to not switch from the use of the stream of first analyte levels for display to use of the stream of second analyte levels for display if the rate of change of the stream of first analyte levels at the first instance of the multiple instances of time is determined to be not less than or equal to the falling rate of change threshold.

In some aspects, comparing the one or more of the first analyte levels to the one or more of the second analyte levels may further include determining whether the difference between the first and second analyte levels for the first instance of the multiple instances of time is less than a minimum analyte level difference threshold. In some aspects, determining whether to switch from use of the stream of first analyte levels for display to use of the stream of second analyte levels for display may include determining to not switch from the use of the stream of first analyte levels for display to use of the stream of second analyte levels for display if the difference between the first and second analyte levels for the first instance of the multiple instances of time is determined to be less than the minimum analyte level difference threshold. In some aspects, the minimum analyte level difference threshold may be 40 mg/dL.

In some aspects, comparing the one or more of the first analyte levels to the one or more of the second analyte levels further may include comparing a minimum cost of the stream of first analyte levels at a last reference analyte value to a minimum cost of the stream of second analyte levels at the last reference analyte value. In some aspects, determining whether to switch from use of the stream of first analyte levels for display to use of the stream of second analyte levels for display may include determining to not switch from the use of the stream of first analyte levels for display to use of the stream of second analyte levels for display if the minimum cost of the stream of second analyte levels at the last reference analyte value is not less than a sum of the minimum cost of the stream of first analyte levels at the last reference analyte value and a minimum cost difference threshold.

In some aspects, determining whether to switch from use of the stream of first analyte levels for display to use of the stream of second analyte levels for display may further include determining whether a minimum cost of the stream of second analyte levels at the last reference analyte value is less than a maximum cost threshold. In some aspects, determining whether to switch from use of the stream of first analyte levels for display to use of the stream of second analyte levels for display may include determining to not switch from the use of the stream of first analyte levels for display to use of the stream of second analyte levels for display if the minimum cost of the stream of second analyte levels at the last reference analyte value is determined to be not less than the maximum cost threshold.

In some aspects, the method may further include, subsequent to switching to use of the stream of second analyte levels for display, determining whether the first analyte level for a second instance of the multiple instances of time is greater than the first analyte level threshold. In some aspects, the method may further include, subsequent to switching to use of the stream of second analyte levels for display, switching to use of the stream of first analyte levels for display if the first analyte level for the second instance of the multiple instances of time is determined to be greater than the first analyte level threshold.

In some aspects, the method may further include, subsequent to switching to use of the stream of second analyte levels for display, determining whether the second analyte level for a second instance of the multiple instances of time is greater than the first analyte level threshold. In some aspects, the method may further include, subsequent to switching to use of the stream of second analyte levels for display, switching to use of the stream of first analyte levels for display if the second analyte level for the second instance of the multiple instances of time is determined to be greater than the first analyte level threshold.

In some aspects, the method may further include, subsequent to switching to use of the stream of second analyte levels for display, determining whether the first analyte level for a second instance of the multiple instances of time is greater than the first analyte level threshold. In some aspects, the method may further include, subsequent to switching to use of the stream of second analyte levels for display, determining whether a rate of change of the stream of first analyte levels at the second instance of the multiple instances of time is greater than or equal to a rising rate of change threshold. In some aspects, the method may further include, subsequent to switching to use of the stream of second analyte levels for display, switching to use of the stream of first analyte levels for display if (i) the first analyte level for the second instance of the multiple instances of time is determined to be greater than the first analyte level threshold and (ii) the rate of change of the stream of first analyte levels at the second instance of the multiple instances of time is determined to be greater than or equal to the rising rate of change threshold.

In some aspects, the method may further include, subsequent to switching to use of the stream of second analyte levels for display, determining whether the second analyte level for a second instance of the multiple instances of time is greater than the first analyte level threshold. In some aspects, the method may further include, subsequent to switching to use of the stream of second analyte levels for display, determining whether a rate of change of the stream of second analyte levels at the second instance of the multiple instances of time is greater than or equal to a rising rate of change threshold. In some aspects, the method may further include, subsequent to switching to use of the stream of second analyte levels for display, switching to use of the stream of first analyte levels for display if (i) the second analyte level for the second instance of the multiple instances of time is determined to be greater than the first analyte level threshold and (ii) the rate of change of the stream of second analyte levels at the second instance of the multiple instances of time is determined to be greater than or equal to the rising rate of change threshold.

In some aspects, the method may further include, subsequent to switching to use of the stream of second analyte levels for display, determining whether the first analyte level for a second instance of the multiple instances of time is less than a second analyte level threshold. In some aspects, the method may further include, subsequent to switching to use of the stream of second analyte levels for display, if the first analyte level for the second instance of the multiple instances of time is determined to be less than the second analyte level threshold, determining whether the first analyte level for a third instance of the multiple instances of time is greater than the second analyte level threshold. In some aspects, the method may further include, subsequent to switching to use of the stream of second analyte levels for display, switching to use of the stream of first analyte levels for display if the first analyte level for the third instance of the multiple instances of time is determined to be greater than the second analyte level threshold.

In some aspects, the method may further include, subsequent to switching to use of the stream of second analyte levels for display, determining whether the second analyte level for a second instance of the multiple instances of time is less than a second analyte level threshold. In some aspects, the method may further include, subsequent to switching to use of the stream of second analyte levels for display, if the second analyte level for the second instance of the multiple instances of time is determined to be less than the second analyte level threshold, determining whether the second analyte level for a third instance of the multiple instances of time is greater than the second analyte level threshold. In some aspects, the method may further include, subsequent to switching to use of the stream of second analyte levels for display, switching to use of the stream of first analyte levels for display if the second analyte level for the third instance of the multiple instances of time is determined to be greater than the second analyte level threshold.

In some aspects, the method may further include, subsequent to switching to use of the stream of second analyte levels for display, determining whether the first analyte level for a second instance of the multiple instances of time is less than a second analyte level threshold. In some aspects, the method may further include, subsequent to switching to use of the stream of second analyte levels for display, if the first analyte level for the second instance of the multiple instances of time is determined to be less than the second analyte level threshold, (i) determining whether the first analyte level for a third instance of the multiple instances of time is greater than the second analyte level threshold and (ii) determining whether a rate of change of the stream of first analyte levels at the third instance of the multiple instances of time is greater than or equal to a rising rate of change threshold. In some aspects, the method may further include, subsequent to switching to use of the stream of second analyte levels for display, switching to use of the stream of first analyte levels for display if (i) the first analyte level for the third instance of the multiple instances of time is determined to be greater than the second analyte level threshold and (ii) the rate of change of the stream of first analyte levels at the third instance of the multiple instances of time is determined to be greater than or equal to the rising rate of change threshold.

In some aspects, the method may further include, subsequent to switching to use of the stream of second analyte levels for display, determining whether the second analyte level for a second instance of the multiple instances of time is less than a second analyte level threshold. In some aspects, the method may further include, subsequent to switching to use of the stream of second analyte levels for display, if the second analyte level for the second instance of the multiple instances of time is determined to be less than the second analyte level threshold, (i) determining whether the second analyte level for a third instance of the multiple instances of time is greater than the second analyte level threshold and (ii) determining whether a rate of change of the stream of second analyte levels at the third instance of the multiple instances of time is greater than or equal to a rising rate of change threshold. In some aspects, the method may further include, subsequent to switching to use of the stream of second analyte levels for display, switching to use of the stream of first analyte levels for display if (i) the second analyte level for the third instance of the multiple instances of time is determined to be greater than the second analyte level threshold and (ii) the rate of change of the stream of second analyte levels at the third instance of the multiple instances of time is determined to be greater than or equal to the rising rate of change threshold.

In some aspects, the second analyte level threshold may be 70 mg/dL. In some aspects, the second analyte level threshold may be 75 mg/dL.

In some aspects, the first analyte level threshold may be 100 mg/dL. In some aspects, the first analyte level threshold may be 110 mg/dL. In some aspects, the maximum analyte level difference threshold may be 40 mg/dL.

In some aspects, switching to use of the stream of second analyte levels for display may include using an average of the first and second analyte levels for one or more transitionary instances of the multiple instances of time for display and then using the second analyte levels for one or more instances of the multiple instances of time subsequent to the one or more transitionary instances.

In some aspects, switching to use of the stream of first analyte levels for display may include using an average of the first and second analyte levels for one or more transitionary instances of the multiple instances of time for display and then using the first analyte levels for one or more instances of the multiple instances of time subsequent to the one or more transitionary instances.

Another aspect of the invention may provide an apparatus including an antenna and processing circuitry. The antenna may be configured to receive sensor data for multiple instances of time, and the sensor data may be conveyed by an analyte sensor. The processing circuitry may be configured to use a first method to calculate a stream of first analyte levels for the multiple instances of time based on at least the sensor data for the multiple instances of time. The processing circuitry may be configured to use a second method to calculate a stream of second analyte levels for the multiple instances of time based on at least the sensor data for the multiple instances of time. The processing circuitry may be configured to use the stream of first analyte levels for display. The processing circuitry may be configured to determine whether to switch from use of the stream of first analyte levels for display to use of the stream of second analyte levels for display. Determining whether to switch from use of the stream of first analyte levels for display to use of the stream of second analyte levels may include comparing one or more of the first analyte levels to one or more of the second analyte levels. The processing circuitry may be configured to continue to use the stream of first analyte levels for display if determined to not switch from use of the stream of first analyte levels to use of the stream of second analyte levels. The processing circuitry may be configured to switch to use of the stream of second analyte levels for display if determined to switch from use of the stream of first analyte levels to use of the stream of second analyte levels.

In some aspects, the first method may be one of a ratio method and a two-parameter method, and the second method may be another of the ratio method and the two-parameter method. In some aspects, the apparatus may further include a display, and the processing circuitry may be further configured to, in using the stream of first analyte levels for display, cause the display to display the one or more of the first analyte levels. In some aspects, the antenna is a first antenna, the apparatus further comprises a second antenna, and the processing circuitry may be further configured to, in using the stream of first analyte levels for display, cause the second antenna to convey one or more of the first analyte levels to a display device for display by the display device.

In some aspects, the processing circuitry may be further configured to receive a first reference analyte value for a first reference instance of time and receive a second reference analyte value for a second reference instance of time, and the processing circuitry may be configured to perform determining whether to switch from use of the stream of first analyte levels for display to use of the stream of second analyte levels for display if the second reference analyte value is received. In some aspects, in comparing the one or more of the first analyte levels to the one or more of the second analyte levels, the processing circuitry may be further configured to: (i) determine an aggregate value of the first analyte levels that are for instances of the multiple instances of time that are between the first and second reference instances of time; (ii) determine an aggregate value of the second analyte levels that are for instances of the multiple instances of time that are between the first and second reference instances of time; and (iii) determine whether the aggregate value of the second analyte levels is lower than the aggregate value of the first analyte levels. In some aspects, the processing circuitry may be further configured to, in determining whether to switch from use of the stream of first analyte levels for display to use of the stream of second analyte levels for display, determine to not switch from the use of the stream of first analyte levels for display to use of the stream of second analyte levels for display if the aggregate value of the second analyte levels is not determined to be lower than the aggregate value of the first analyte levels. In some aspects, the processing circuitry may be further configured to, in determining whether to switch from use of the stream of first analyte levels for display to use of the stream of second analyte levels for display, determine to switch from the use of the stream of first analyte levels for display to use of the stream of second analyte levels for display if the aggregate value of the second analyte levels is determined to be lower than the aggregate value of the first analyte levels.

In some aspects, the determined aggregate value of the first analyte levels that are for the instances of the multiple instances of time that are between the first and second reference instances of time may be a first quartile of the first analyte levels that are for the instances of the multiple instances of time that are between the first and second reference instances of time, and the determined aggregate value of the second analyte levels that are for the instances of the multiple instances of time that are between the first and second reference instances of time may be a first quartile of the second analyte levels that are for the instances of the multiple instances of time that are between the first and second reference instances of time.

In some aspects, the apparatus may be further configured to receive a third reference analyte value for a third reference instance of time; and the processing circuitry may be further configured to: (i) determine a second aggregate value of the first analyte levels that are for instances of the multiple instances of time that are between the second and third reference instances of time; (ii) determine a second aggregate value of the second analyte levels that are for instances of the multiple instances of time that are between the second and third reference instances of time; (iii) determine whether the second aggregate value of the second analyte levels is lower than the second aggregate value of the first analyte levels; (iv) use the stream of first analyte levels for display for one or more instances of the multiple instances of time that are subsequent to the third reference instances of time if the second aggregate value of the second analyte levels is not determined to be lower than the second aggregate value of the first analyte levels; and (v) use the stream of second analyte levels for display for the one or more instances of the multiple instances of time that are subsequent to the third reference instances of time if the second aggregate value of the second analyte levels is determined to be lower than the second aggregate value of the first analyte levels.

In some aspects, in determining whether to switch from use of the stream of first analyte levels for display to use of the stream of second analyte levels, the processing circuitry may be configured to: (a) determine whether the first analyte level for a first instance of the multiple instances of time is less than a first analyte level threshold and/or (b) determine at the first instance of the multiple instances of time whether a predicted first analyte level is less than the first analyte level threshold. In some aspects, in comparing the one or more of the first analyte levels to the one or more of the second analyte levels, the processing circuitry may be further configured to determine whether the second analyte level for a first instance of the multiple instances of time is lower than the first analyte level for the first instance of the multiple instances of time. In some aspects, in comparing the one or more of the first analyte levels to the one or more of the second analyte levels, the processing circuitry may be further configured to determine whether a difference between the first and second analyte levels for the first instance of the multiple instances of time is greater than a maximum analyte level difference threshold. In some aspects, the processing circuitry may be further configured to, in determining whether to switch from use of the stream of first analyte levels for display to use of the stream of second analyte levels, determine whether the first analyte level for the first instance of the multiple instances of time is less than the first analyte level threshold.

In some aspects, the processing circuitry may be configured to, in determining whether to switch from use of the stream of first analyte levels for display to use of the of the stream of second analyte levels for display, determine at the first instance of the multiple instances of time whether the predicted first analyte level is less than the first analyte level threshold. In some aspects, the processing circuitry may be further configured to, in determining whether to switch from use of the stream of first analyte levels for display to use of the stream of second analyte levels for display, determine to not switch from the use of the stream of first analyte levels for display to use of the stream of second analyte levels for display if (a) the first analyte level for the first instance of the multiple instances of time is less than the second analyte level for the first instance of time and/or (b) the difference between the first and second analyte levels for the first instance of time is greater than the maximum analyte level difference threshold. In some aspects, the processing circuitry may be configured to, in determining whether to switch from use of the stream of first analyte levels for display to use of the stream of second analyte levels for display, determine to switch from the use of the stream of first analyte levels for display to use of the stream of second analyte levels for display if (i) the first analyte level for the first instance of time is greater than the second analyte level for the second instance of time and (ii) the difference between the first and second analyte levels for the first instance of time is less than the analyte difference threshold.

In some aspects, the processing circuitry may be configured to, in determining whether to switch from use of the stream of first analyte levels for display to use of the stream of second analyte levels for display, determine whether a rate of change of the stream of first analyte levels at the first instance of the multiple instances of time is less than or equal to a falling rate of change threshold. In some aspects, the processing circuitry may be configured to, in determining whether to switch from use of the stream of first analyte levels for display to use of the stream of second analyte levels for display, determine to not switch from the use of the stream of first analyte levels for display to use of the stream of second analyte levels for display if the rate of change of the stream of first analyte levels at the first instance of the multiple instances of time is determined to be not less than or equal to the falling rate of change threshold.

In some aspects, the processing circuitry may be configured to, in comparing the one or more of the first analyte levels to the one or more of the second analyte levels, determine whether the difference between the first and second analyte levels for the first instance of the multiple instances of time is less than a minimum analyte level difference threshold. In some aspects, the processing circuitry may be configured to, in determining whether to switch from use of the stream of first analyte levels for display to use of the stream of second analyte levels for display, determine to not switch from the use of the stream of first analyte levels for display to use of the stream of second analyte levels for display if the difference between the first and second analyte levels for the first instance of the multiple instances of time is determined to be less than the minimum analyte level difference threshold. In some aspects, the minimum analyte level difference threshold is 40 mg/dL.

In some aspects, the processing circuitry may be configured to, in comparing the one or more of the first analyte levels to the one or more of the second analyte levels, compare a minimum cost of the stream of first analyte levels at a last reference analyte value to a minimum cost of the stream of second analyte levels at the last reference analyte value. In some aspects, the processing circuitry may be configured to, in determining whether to switch from use of the stream of first analyte levels for display to use of the stream of second analyte levels for display, determine to not switch from the use of the stream of first analyte levels for display to use of the stream of second analyte levels for display if the minimum cost of the stream of second analyte levels at the last reference analyte value is not less than a sum of the minimum cost of the stream of first analyte levels at the last reference analyte value and a minimum cost difference threshold.

In some aspects, the processing circuitry may be configured to, in determining whether to switch from use of the stream of first analyte levels for display to use of the stream of second analyte levels for display, determine whether a minimum cost of the stream of second analyte levels at the last reference analyte value is less than a maximum cost threshold. In some aspects, the processing circuitry may be configured to, in determining whether to switch from use of the stream of first analyte levels for display to use of the stream of second analyte levels for display, determine to not switch from the use of the stream of first analyte levels for display to use of the stream of second analyte levels for display if the minimum cost of the stream of second analyte levels at the last reference analyte value is determined to be not less than the maximum cost threshold.

In some aspects, the processing circuitry may be further configured to, subsequent to switching to use of the stream of second analyte levels for display: (i) determine whether the first analyte level for a second instance of the multiple instances of time is greater than the first analyte level threshold; and (ii) switch to use of the stream of first analyte levels for display if the first analyte level for the second instance of the multiple instances of time is determined to be greater than the first analyte level threshold.

In some aspects, the processing circuitry may be further configured to, subsequent to switching to use of the stream of second analyte levels for display: (i) determine whether the second analyte level for a second instance of the multiple instances of time is greater than the first analyte level threshold; and (ii) switch to use of the stream of first analyte levels for display if the second analyte level for the second instance of the multiple instances of time is determined to be greater than the first analyte level threshold.

In some aspects, the processing circuitry may be further configured to, subsequent to switching to use of the stream of second analyte levels for display: (1) determine whether the first analyte level for a second instance of the multiple instances of time is greater than the first analyte level threshold; (2) determine whether a rate of change of the stream of first analyte levels at the second instance of the multiple instances of time is greater than or equal to a rising rate of change threshold; and (3) switch to use of the stream of first analyte levels for display if (i) the first analyte level for the second instance of the multiple instances of time is determined to be greater than the first analyte level threshold and (ii) the rate of change of the stream of first analyte levels at the second instance of the multiple instances of time is determined to be greater than or equal to the rising rate of change threshold.

In some aspects, the processing circuitry may be further configured to, subsequent to switching to use of the stream of second analyte levels for display, (1) determine whether the second analyte level for a second instance of the multiple instances of time is greater than the first analyte level threshold; (2) determine whether a rate of change of the stream of second analyte levels at the second instance of the multiple instances of time is greater than or equal to a rising rate of change threshold; and (3) switch to use of the stream of first analyte levels for display if (i) the second analyte level for the second instance of the multiple instances of time is determined to be greater than the first analyte level threshold and (ii) the rate of change of the stream of second analyte levels at the second instance of the multiple instances of time is determined to be greater than or equal to the rising rate of change threshold.

In some aspects, the processing circuitry may be further configured to, subsequent to switching to use of the stream of second analyte levels for display: (1) determine whether the first analyte level for a second instance of the multiple instances of time is less than a second analyte level threshold; (2) if the first analyte level for the second instance of the multiple instances of time is determined to be less than the second analyte level threshold, determine whether the first analyte level for a third instance of the multiple instances of time is greater than the second analyte level threshold; and (3) switch to use of the stream of first analyte levels for display if the first analyte level for the third instance of the multiple instances of time is determined to be greater than the second analyte level threshold.

In some aspects, the processing circuitry may be further configured to, subsequent to switching to use of the stream of second analyte levels for display: (1) determine whether the second analyte level for a second instance of the multiple instances of time is less than a second analyte level threshold; (2) if the second analyte level for the second instance of the multiple instances of time is determined to be less than the second analyte level threshold, determine whether the second analyte level for a third instance of the multiple instances of time is greater than the second analyte level threshold; and (3) switch to use of the stream of first analyte levels for display if the second analyte level for the third instance of the multiple instances of time is determined to be greater than the second analyte level threshold.

In some aspects, the processing circuitry may be further configured to, subsequent to switching to use of the stream of second analyte levels for display: (1) determine whether the first analyte level for a second instance of the multiple instances of time is less than a second analyte level threshold; (2) if the first analyte level for the second instance of the multiple instances of time is determined to be less than the second analyte level threshold, (i) determine whether the first analyte level for a third instance of the multiple instances of time is greater than the second analyte level threshold and (ii) determine whether a rate of change of the stream of first analyte levels at the third instance of the multiple instances of time is greater than or equal to a rising rate of change threshold; and (3) switch to use of the stream of first analyte levels for display if (i) the first analyte level for the third instance of the multiple instances of time is determined to be greater than the second analyte level threshold and (ii) the rate of change of the stream of first analyte levels at the third instance of the multiple instances of time is determined to be greater than or equal to the rising rate of change threshold.

In some aspects, the processing circuitry may be further configured to, subsequent to switching to use of the stream of second analyte levels for display: (1) determine whether the second analyte level for a second instance of the multiple instances of time is less than a second analyte level threshold; (2) if the second analyte level for the second instance of the multiple instances of time is determined to be less than the second analyte level threshold, (i) determine whether the second analyte level for a third instance of the multiple instances of time is greater than the second analyte level threshold and (ii) determine whether a rate of change of the stream of second analyte levels at the third instance of the multiple instances of time is greater than or equal to a rising rate of change threshold; and (3) switch to use of the stream of first analyte levels for display if (i) the second analyte level for the third instance of the multiple instances of time is determined to be greater than the second analyte level threshold and (ii) the rate of change of the stream of second analyte levels at the third instance of the multiple instances of time is determined to be greater than or equal to the rising rate of change threshold.

In some aspects, the second analyte level threshold may be 70 mg/dL. In some aspects, the second analyte level threshold may be 75 mg/dL.

In some aspects, the first analyte level threshold may be 100 mg/dL. In some aspects, the first analyte level threshold may be 110 mg/dL. In some aspects, the maximum analyte level difference threshold may be 40 mg/dL

In some aspects, the processing circuitry, in switching to use of the stream of second analyte levels for display, may be configured to use an average of the first and second analyte levels for one or more transitionary instances of the multiple instances of time for display and then using the second analyte levels for one or more instances of the multiple instances of time subsequent to the one or more transitionary instances.

In some aspects, the processing circuitry, in switching to use of the stream of second analyte levels for display, may be configured to use an average of the first and second analyte levels for one or more transitionary instances of the multiple instances of time for display and then use the first analyte levels for one or more instances of the multiple instances of time subsequent to the one or more transitionary instances.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting aspects of the present invention. In the drawings, like reference numbers indicate identical or functionally similar elements.

FIG. 1 is a schematic view illustrating an exemplary analyte monitoring system according to some aspects.

FIG. 2 is a schematic view illustrating an exemplary analyte sensor of the analyte monitoring system according to some aspects.

FIG. 3 is a schematic view illustrating an exemplary transceiver of the analyte monitoring system according to some aspects.

FIG. 4 is a schematic view illustrating an exemplary display device of the analyte monitoring system according to some aspects.

FIG. 5 is a schematic view illustrating exemplary processing circuitry of the analyte monitoring system according to some aspects.

FIG. 6 is a flowchart illustrating a process according to some aspects.

FIG. 7 is a flowchart illustrating a first dynamic stream selection (DSS) process according to some aspects.

FIG. 8A is a graph illustrating an example of the first DSS process according to some aspects. FIGS. 8B and 8C show enlarged left and right halves, respectively, of the graph shown in FIG. 8A.

FIG. 9A is a flowchart illustrating a second DSS process according to some aspects.

FIG. 9B is a flowchart illustrating a third DSS process according to some aspects.

FIG. 9C is a flowchart illustrating a third DSS process according to some aspects.

FIG. 9D is a flowchart illustrating a fourth DSS process according to some aspects.

FIGS. 10A-10E illustrate examples of the second through fourth DSS processes according to some aspects.

FIGS. 11A and 11B illustrate alternative examples of switching to use of the stream of second analyte levels for display according to some aspects.

FIG. 12 illustrates a block diagram illustrating exemplary processing circuitry of the analyte monitoring system according to some aspects.

### DETAILED DESCRIPTION OF PREFERRED EMBODIMENTS

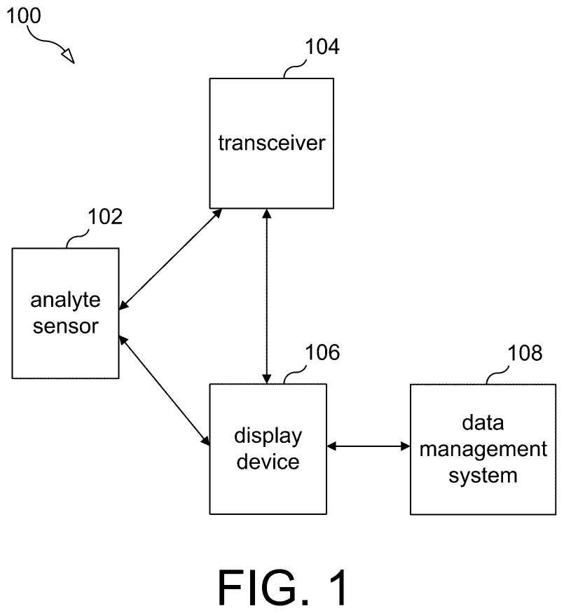

FIG. 1 is a schematic view of an exemplary analyte monitoring system 100 embodying aspects of the present invention. In some aspects, as shown in FIG. 1, the analyte monitoring system 100 may be a continuous analyte monitoring system (e.g., a continuous glucose monitoring system). In some aspects, the analyte monitoring system 100 may include an analyte sensor 102, a transceiver 104, a display device 106, and/or a data management system (DMS) 108 hosted by a remote server or network attached storage hardware.

In some aspects, the sensor 102 may be small, fully subcutaneously implantable sensor measures analyte (e.g., glucose) concentrations in a medium (e.g., interstitial fluid) of a living animal (e.g., a living human). However, this is not required, and, in some alternative aspects, the analyte sensor 102 may be a partially implantable (e.g., transcutaneous) sensor or a fully external sensor. In some aspects, the analyte sensor 102 may be powered by (a) one or more charge storage devices (e.g., one or more batteries) included in the analyte sensor 102 and/or (b) power received from a source (e.g., the transceiver 104 and/or the display device 106) external to the analyte sensor 102. In some non-limiting aspects, the analyte sensor 102 may include one or more optical sensors (e.g., one or more fluorometers). In some aspects, the analyte sensor 102 may be a chemical or biochemical sensor. In some aspects, the analyte sensor 102 may be a radio frequency identification (RFID) device.

In some aspects, the transceiver 104 may be an externally worn transceiver (e.g., attached via an armband, wristband, waistband, or adhesive patch). In some aspects, the transceiver 104 may remotely power and/or communicate with the sensor to initiate and receive the measurements (e.g., via near field communication (NFC) or far field communication). However, this is not required, and, in some alternative aspects, the transceiver 104 may power and/or communicate with the sensor 102 via one or more wired connections. In some aspects, the transceiver 104 may be a smartphone (e.g., an NFC-enabled smartphone). In some aspects, the transceiver 104 may communicate information (e.g., one or more analyte concentrations and/or one or more sensor measurements) wirelessly (e.g., via a Bluetooth™ communication standard such as, for example and without limitation Bluetooth Low Energy) to a mobile medical application (MMA) running on a display device 106 (e.g., a smartphone such as, for example, an NFC-enabled smartphone).

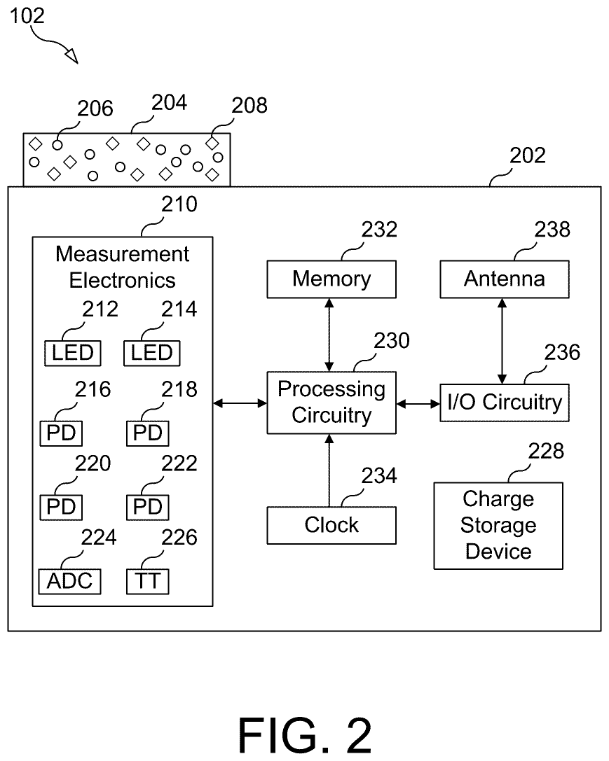

FIG. 2 illustrates an exemplary aspect in which the analyte sensor 102 of the analyte monitoring system 100 is a fully implantable electro-optical sensor. However, this is not required, and, in some alternative aspects, the analyte sensor 102 may be a different type of analyte sensor (e.g., a transcutaneous electrochemical sensor). In some aspects, as shown in FIG. 2, the analyte sensor 102 may include a sensor housing 202 (i.e., body, shell, capsule, or encasement), which may be rigid and biocompatible. In some aspects, the sensor housing 202 may be a silicon tube. However, this is not required, and, in other aspects, different materials and/or shapes may be used for the sensor housing 202. In some aspects, the analyte sensor 102 may include a transmissive optical cavity (e.g., within the sensor housing 202). In some aspects, the transmissive optical cavity may be formed from a suitable, optically transmissive polymer material, such as, for example, acrylic polymers (e.g., polymethylmethacrylate (PMMA)). However, this is not required, and, in other aspects, different materials may be used for the transmissive optical cavity.

In some aspects, the analyte sensor 102 may include one or more analyte and/or interferent indicators 204, which may be, for example, polymer grafts or hydrogels coated, diffused, adhered, embedded, or grown on or in one or more portions of the exterior surface of the sensor housing 202. In some aspects, the one or more analyte and/or interferent indicators 204, may be porous and may allow the analyte (e.g., glucose) in a medium (e.g., interstitial fluid) to diffuse into the one or more analyte and/or interferent indicators 204.

In some aspects, as shown in FIG. 2, the one or more analyte and/or interferent indicators 204 may include analyte indicator molecules 206 and/or interferent indicator molecules 208 (e.g., degradation indicator molecules). In some aspects, analyte sensor 102 may use the analyte indicator molecules 206 to measure the presence, amount, and/or concentration of an analyte (e.g., glucose, oxygen, cardiac markers, low-density lipoprotein (LDL), high-density lipoprotein (HDL), or triglycerides). In some aspects, the analyte indicator molecules 206 may use the interferent indicator molecules 208 to measure in vivo (e.g., ROS induced) signal degradation. In some aspects, in the one or more analyte and/or interferent indicators 204, the analyte indicator molecules 206 and/or the interferent indicator molecules 208 may be copolymerized into a single biocompatible hydrogel. In some aspects, the analyte indicator molecules 206 and/or the interferent indicator molecules 208 may have negligible spectral overlap and undergo similar degradation (e.g., similar degradation of boronic acids) in vivo.

In some aspects, the analyte indicator molecules 206 may have one or more detectable properties (e.g., optical properties) that vary in accordance with (i) the amount or concentration of the analyte in proximity to the analyte and/or interferent indicator 204 and (ii) an effect on the analyte indicator molecules 206 (e.g., changes to the analyte indicator molecules 206). In some aspects, the changes to the analyte indicator molecules 206 may comprise the extent to which the analyte indicator molecules 206 have degraded. In some aspects, the degradation may be (at least in part) ROS-induced oxidation. In some aspects, the analyte indicator molecules 206 may be fluorescent analyte indicator molecules. In some aspects, the analyte indicator molecules 206 may be distributed throughout the analyte and/or interferent indicator 204. In some aspects, the analyte indicator molecules 206 may be phenylboronic-based analyte indicator molecules. However, a phenylboronic-based analyte indicator is not required, and, in some alternative aspects, the analyte sensor 102 may include different analyte indicator molecules, such as, for example and without limitation, glucose oxidase-based indicators, glucose dehydrogenase-based indicators, and glucose binding protein-based indicators.

In some aspects, the interferent indicator molecules 208 may have one or more detectable properties (e.g., optical properties) that vary in accordance with changes to the interferent indicator molecules 208. In some aspects, the interferent indicator molecules 208 are not sensitive to the amount of concentration of the analyte in proximity to the analyte and/or interferent indicator 204. That is, in some aspects, the one or more detectable properties of the interferent indicator molecules 208 do not vary in accordance with the amount or concentration of the analyte in proximity to the analyte and/or interferent indicator 204. However, this is not required, and, in some alternative aspects, the one or more detectable properties of interferent indicator molecules 208 may vary in accordance with the amount or concentration of the analyte in proximity to the analyte and/or interferent indicator 204.

In some aspects, the changes to the interferent indicator molecules 208 may comprise the extent to which the interferent indicator molecules 208 have degraded. In some aspects, the degradation may be (at least in part) ROS-induced oxidation. In some aspects, the interferent indicator molecules 208 may be fluorescent interferent indicator molecules. In some aspects, the interferent indicator molecules 208 may be distributed throughout the analyte and/or interferent indicator 204. In some aspects, the interferent indicator molecules 208 may be phenylboronic-based interferent indicator molecules. However, phenylboronic-based interferent indicator molecules are not required, and, in some alternative aspects, the analyte sensor 102 may include different interferent indicator molecules 208, such as, for example and without limitation, amplex red-based interferent indicator molecules, dichlorodihydrofluorescein-based interferent indicator molecules, dihydrorhodamine-based interferent indicator molecules, and scopoletin-based interferent indicator molecules.

In some aspects, the analyte sensor 102 may measure changes to the analyte indicator molecules 206 of an analyte and/or interferent indicator 1304 indirectly using the interferent indicator molecules 208 of the analyte and/or interferent indicator 204, which may by sensitive to degradation by reactive oxygen species (ROS) but not sensitive to the analyte. In some aspects, the interferent indicator molecules 208 may have one or more optical properties that change with extent of oxidation and may be used as a reference for measuring and correcting for extent of oxidation of the analyte indicator molecules 206. In some aspects, the extent to which the interferent indicator molecules 208 have degraded may correspond to the extent to which the analyte indicator molecules 206 have degraded. For example, in aspects, the extent to which the interferent indicator molecules 208 have degraded may be proportional to the extent to which the analyte indicator molecules 206 have degraded. In some aspects, the extent to which the analyte indicator molecules 206 have degraded may be calculated based on the extent to which the interferent indicator molecules 208 have degraded. In some aspects, the analyte monitoring system 100 may correct for changes in the analyte indicator molecules 206 using an empiric correlation established through laboratory testing.

In some aspects, the analyte sensor 102 may include measurement electronics 210 (e.g., optical measurement electronics). In some aspects, the measurement electronics 210 may include one or more light sources and/or one or more photodetectors. For example, in some aspects, as shown in FIG. 2, the measurement electronics 210 may include one or more first light sources 212 that emit first excitation light over a wavelength range that interacts with the analyte indicator molecules 206 in the analyte and/or interferent indicator 204. In some aspects, the first excitation light may be ultraviolet (UV) light. In some aspects, the analyte sensor 102 may include one or more second light sources 214 that emit second excitation light over a wavelength range that interacts with the interferent indicator molecules 208 in the analyte and/or interferent indicator 204. In some aspects, the second excitation light may be, for example and without limitation, blue light.

In some aspects, an analyte (e.g., glucose) may bind reversibly to some of the analyte indicator molecules 206, the analyte indicator molecules 206 to which the analyte is bound may emit first emission light (e.g., fluorescent light) when irradiated by the first excitation light, and the analyte indicator molecules 206 to which the analyte is not bound may not emit light (or emit only a small amount of light) when irradiated by the first excitation light. In some aspects, oxidation of the interferent indicator molecules 208 may cause the interferent indicator molecules 208 to emit second emission light (e.g., when irradiated by the second excitation light). In some aspects, oxidation of the interferent indicator molecules 208 may additionally or alternatively cause the absorption of the interferent indicator molecules 208 (e.g., absorption of the second excitation light by the interferent indicator molecules 208) to change.

In some aspects, as shown in FIG. 2, the measurement electronics 210 of the analyte sensor 102 may also include one or more photodetectors 216, 218, and 220 (e.g., photodiodes, phototransistors, photoresistors, or other photosensitive elements). In some aspects, the measurement electronics 210 of the analyte sensor 102 may include one or more signal photodetectors 216 sensitive to first emission light (e.g., fluorescent light) emitted by the analyte indicator molecules 206 such that a signal generated by a signal photodetector 216 is indicative of the level of first emission light of the analyte indicator molecules 206 and, thus, the amount of analyte of interest (e.g., glucose). In some aspects, the measurement electronics 210 may include one or more reference photodetectors 218 sensitive to first excitation light that may be reflected from the analyte and/or interferent indicator 204 such that a signal generated by a photodetector 218 in response thereto is indicative of the level of reflected first excitation light. In some aspects, the analyte sensor 102 may include one or more interferent photodetectors 220 sensitive to second emission light (e.g., fluorescent light) emitted by the interferent indicator molecules 208 such that a signal generated by an interferent photodetector 220 in response thereto that is indicative of the level of second emission light of the interferent indicator molecules 208 and, thus, the amount of degradation (e.g., oxidation). In some aspects, the one or more signal photodetectors 216 may be sensitive to second excitation light that may be reflected from the analyte and/or interferent indicator 204. In this way, the one or more signal photodetectors 216 may act as reference photodetectors when the one or more second light sources 214 are emitting second excitation light.

However, it is not required that the one or more signal photodetectors 224 act as reference photodetectors when the one or more second light sources 214 are emitting second excitation light. In some alternative aspects, as shown in FIG. 2, the measurement electronics 210 of the analyte sensor 102 may include one or more second reference photodetectors 222 that act as reference photodetectors when the one or more second light sources 214 are emitting second excitation light. In some aspects, the one or more second reference photodetectors 222 may be sensitive to second excitation light that may be reflected from the analyte and/or interferent indicator 204 such that a signal generated by a second reference photodetector 222 in response thereto is indicative of the level of reflected second excitation light.

In some aspects, one or more of the photodetectors 216, 218, 220, 222 may be covered by one or more filters that allow only a certain subset of wavelengths of light to pass through and reflect (or absorb) the remaining wavelengths. In some aspects, one or more filters on the one or more signal photodetectors 216 may allow only a subset of wavelengths corresponding to first emission light and/or the reflected second excitation light. In some aspects, one or more filters on the one or more reference photodetectors 218 may allow only a subset of wavelengths corresponding to the reflected first excitation light. In some aspects, one or more filters on the one or more interferent photodetectors 220 may allow only a subset of wavelengths corresponding to second emission light. In some aspects in which the analyte sensor 102 includes one or more second reference photodetectors 222, one or more filters on the one or more second reference photodetectors 222 may allow only a subset of wavelengths corresponding to the reflected second excitation light.

In some aspects, as shown in FIG. 2, the measurement electronics 210 of the analyte sensor 102 may include one or more temperature transducers 226. In some aspects, the measurement electronics 210 may include one or more light source drivers, one or more amplifiers, one or more analog-to-digital convertors (ADCs) 224, one or more comparators, and/or one or more multiplexors. In some aspects, the one or more ADCs 224 may convert analog signals output by the photodetectors 216, 218, 220, 222 and/or one or more temperature transducers 226 to digital signals.

In some aspects, as shown in FIG. 2, the analyte sensor 102 may include a charge storage device 228, processing circuitry 230, a memory 232, a clock 234, an input/output (I/O) circuit 236, and/or an antenna 238. In some aspects, the I/O circuit 236 may include I/O digital circuitry and/or I/O analog circuitry. In some aspects, the antenna 238 may be electrically connected to the I/O circuit 236, which may use current flowing through the antenna 238 to generate power for the sensor 102 and/or to extract data from the current. In some aspects, the I/O circuit 236 may also convey data (e.g., to the transceiver 104 and/or display device 106) by modulating the current the flowing through the antenna 238. In some aspects, the I/O circuit 236 may be electrically connected to and be powered by the antenna 238 and/or the charge storage device 228. In some aspects, although not shown in FIG. 2, the analyte sensor 102 may include multiple sensing devices, and the antenna 238 may be electrically connected to the circuitry of the multiple sensing devices.

In some aspects, the charge storage device (CSD) 228 may provide power to the clock 234 and to the processing circuitry 230. In some aspects, the CSD-powered clock 234 may provide a continuous clock for driving circuitry of the sensor 102 even when the sensor 102 is not receiving power from an external device (e.g., the transceiver 104 and/or the display device 106). In some aspects, the processing circuitry 230 may use the continuous clock output of the clock 234 to keep track of time and initiate autonomous, self-powered analyte measurements when appropriate (e.g., at periodic intervals, such as, for example, every minute, every two minutes, every 5 minutes, every 10 minutes, every 15 minutes, every half-hour, every hour, every two hours, every six hours, every twelve hours, or every day). In some aspects, the processing circuitry 230 may control the measurement electronics 210 to perform an autonomous analyte measurement sequence, and the results of the autonomous analyte measurement may be stored in the memory 232. In some aspects, the I/O circuit 236 may convey one or more of the stored measurements to the external device (e.g., the transceiver 104 and/or the display device 106) at a later time. For example, in some request aspects, the I/O circuit 236 may convey one or more of the stored measurements in response to the analyte sensor 102 receiving and decoding a measurement data request from the transceiver 104 and/or the display device 106. In some alternative aspects, the I/O circuit 236 may convey one or more of the stored measurements in response to detecting that the transceiver 104 and/or display device 106 is present (e.g., when an electrodynamic field generated by the transceiver 1204 and/or display device 1206 induces a current in the antenna 1324 of the analyte sensor 102). In some aspects in which the analyte sensor 102 include multiple sensing devices, although not shown in FIG. 2, the CSD 228 may be electrically connected to the circuitry of the multiple sensing devices.

In some aspects, the memory 232 may be a nonvolatile storage medium. In some aspects, the memory 232 may be an electrically erasable programmable read only memory (EEPROM). However, in some alternative aspects, other types of nonvolatile storage media, such as flash memory, may be used. In some aspects, the memory 232 may include an address decoder. In some aspects, the memory 232 may store measurement information autonomously generated while the sensor 102 is powered from the charge storage device 228. In some aspects, the memory 232 may additionally or alternatively store one or more time-stamps identifying when the measurement data was generated, sensor calibration data, a unique sensor identification, setup information, and/or integrated circuit calibration data. In some aspects, the unique identification information may, for example, enable full traceability of the sensor 102 through its production and subsequent use.

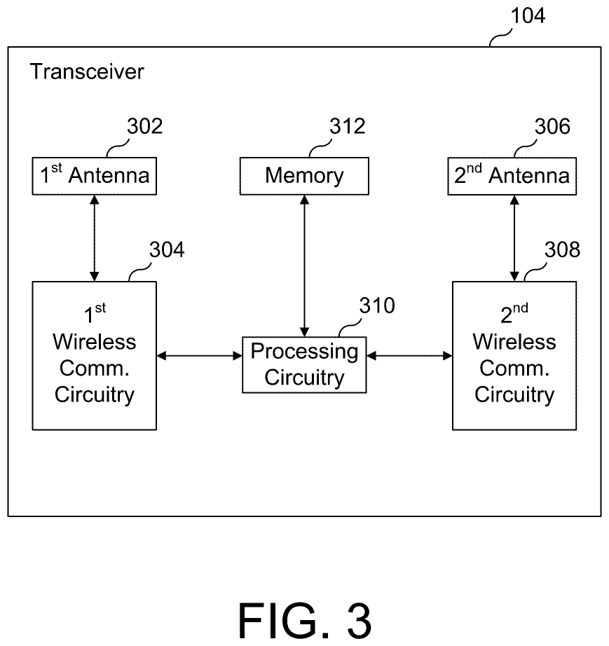

FIG. 3 illustrates an exemplary aspect in which the transceiver 104 of the analyte monitoring system 100 is a wireless transceiver (e.g., a wireless on-body transceiver). However, this is not required, and, in some alternative aspects, the transceiver 104 may be a different type of transceiver (e.g., a transceiver having a wired connection to the analyte sensor 102). In some aspects, as shown in FIG. 3, the transceiver 104 may include a first antenna 302, first wireless communication circuitry 304, a second antenna 306, second wireless communication circuitry 308, processing circuitry 310, and/or a memory 312. In some aspects, the processing circuitry 310 may control the overall operation of the transceiver 104.

In some aspects, the transceiver 104 may include a sensor interface device. In some aspects, the sensor interface device of the transceiver 104 may include the first antenna 302 and the first wireless communication circuitry 304. In some aspects, the first wireless communication circuitry 304 may enable the transceiver 104 to communicate directly with the analyte sensor 102. In some aspects, the transceiver 104 and the sensor 102 may communicate using NFC (e.g. at a frequency of 13.56 MHz). In some aspects, the first antenna 302 of the transceiver 104 may include an inductor (e.g. flat antenna, loop antenna, etc.) that is configured to permit adequate field strength to be achieved when brought within adequate physical proximity to the antenna 238 of the sensor 102.

In some aspects, the transceiver 104 may use the first antenna 302 and the first wireless communication circuitry 304 to receive sensor data from the analyte sensor 102. In some aspects, the received sensor data may be for multiple instances of time. In some aspects, each time that the analyte sensor 102 generates sensor data for an instance of time, the analyte sensor 102 may convey and the transceiver 104 may receive the sensor data for the instance of time (e.g., the transceiver 104 may receive the sensor data as the analyte sensor 102 generates the sensor data). In some alternative aspects, the analyte sensor 102 may store sensor data for multiple instances of time (e.g., in the memory 232) before conveying the sensor data for the multiple instances of time, which may be received by the transceiver 104. In some aspects, the processing circuitry 310 may store the received sensor data in the memory 312. In some aspects, the memory 312 may be non-volatile and/or capable of being electronically erased and/or rewritten. In some aspects, the memory 312 may be, for example and without limitations a Flash memory.

In some aspects, the received sensor data may be for multiple instances of time. In some aspects, the received sensor data may include, for example and without limitation, light measurements and temperature measurements. In some aspects, the received sensor data may include time stamps, which may each indicate an instance of time at which the analyte sensor 102 took one or more of the light measurements and one or more of the temperature measurements. In some aspects, each time stamp may be a count of cycles of the clock 234. However, it is not required that received sensor data includes time stamps, and, in some aspects, the transceiver 104 may add time stamps to the received sensor data (e.g., if the transceiver 104 receives the sensor data as analyte sensor 102 generates the sensor data).

In some aspects, the processing circuitry 310 may use the sensor data to calculate analyte levels (e.g., glucose levels). In some aspects, the processing circuitry 310 may use the sensor data to calculate blood analyte levels. In some aspects in which the processing circuitry 310 calculates blood analyte levels, for each of the instances of time, the processing circuitry 310 may use the sensor data to calculate an interstitial fluid ISF analyte level, may calculate a rate of change of the ISF analyte level, and may calculate a blood analyte level based on the calculated ISF analyte level and the calculated rate of change of the ISF analyte level. In some aspects, the processing circuitry 310 may store the calculated analyte levels in the memory 312. In some alternative aspects, the processing circuitry 310 of the transceiver 104 may not calculate analyte levels.

In some aspects, the transceiver 104 may include a display interface device. In some aspects, the display device interface device may include the second antenna 306 and the second wireless communication circuitry 308. In some aspects, the second wireless communication circuitry 308 may enable wireless communication by the transceiver 104 with one or more external devices, such as, for example, one or more personal computers, one or more other transceivers 104, and/or display devices 106 via the second antenna 306. In some aspects, the second wireless communication circuitry 308 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some aspects, the second antenna 306 may be, for example and without limitation, a Bluetooth antenna.

In some aspects in which the transceiver 104 calculates analyte levels, the transceiver 104 may use the second antenna 306 and the second wireless communication circuitry 308 to convey calculated analyte levels to the display device 106. In some aspects in which the transceiver 104 calculates and conveys analyte levels, the transceiver 104 may additionally convey the sensor data to the display device 106. In some alternative aspects, the transceiver 104 may not calculate analyte levels. In some aspects in which the transceiver 104 does not calculate analyte levels, the transceiver 104 may use the second antenna 306 and the second wireless communication circuitry 308 to convey sensor data to the display device 106, and the display device 106 may use the sensor data to calculate analyte levels.

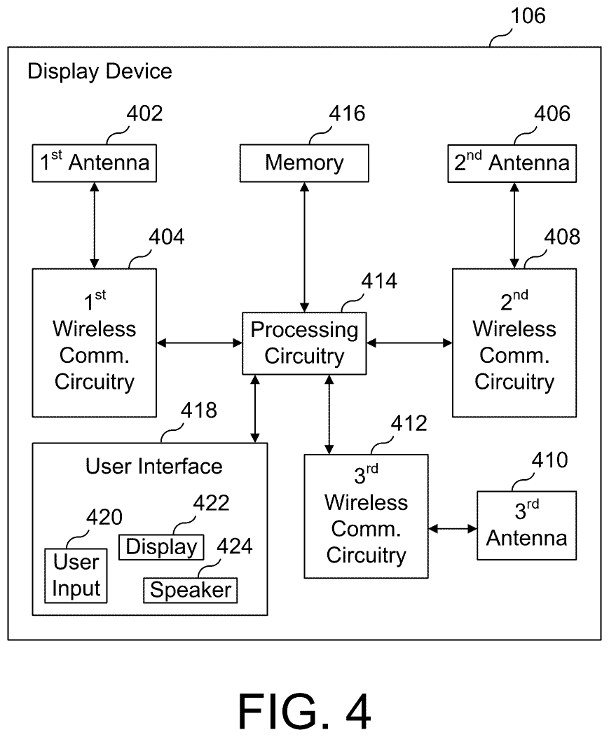

FIG. 4 is a block diagram of the display device 106 of the analyte monitoring system 100 according to some aspects. In some aspects, as shown in FIG. 4, the display device 106 may include a first antenna 402, first wireless communication circuitry 404, a second antenna 406, a second wireless communication circuitry 408, a third antenna 410, a third wireless communication circuitry 412, processing circuitry 414, a memory 416, and/or a user interface 418. In some aspects, the processing circuitry 414 may control the overall operation of the display device 106.

In some aspects, the display device 106 may include a sensor interface device. In some aspects, the sensor interface device of the display device 106 may include the first antenna 402 and the first wireless communication circuitry 404. In some aspects, the first wireless communication circuitry 404 may enable the display device 106 to communicate directly with the analyte sensor 102. In some aspects, the display device 106 and the sensor 102 may communicate using NFC (e.g. at a frequency of 13.56 MHz). In some aspects, the first antenna 402 of the display device 106 may include an inductor (e.g. flat antenna, loop antenna, etc.) that is configured to permit adequate field strength to be achieved when brought within adequate physical proximity to the antenna 238 of the sensor 102.

In some aspects, the display device 106 may use the first antenna 402 and the first wireless communication circuitry 404 to receive sensor data from the analyte sensor 102. In some aspects, the received sensor data may be for multiple instances of time. In some aspects, each time that the analyte sensor 102 generates sensor data for an instance of time, the analyte sensor 102 may convey and the display device 106 may receive the sensor data for the instance of time (e.g., the display device 106 may receive the sensor data as the analyte sensor 102 generates the sensor data). In some alternative aspects, the analyte sensor 102 may store sensor data for multiple instances of time (e.g., in the memory 232) before conveying the sensor data for the multiple instances of time, which may be received by the display device 106. In some aspects, the processing circuitry 414 may store the received sensor data in the memory 416. In some aspects, the memory 416 may be non-volatile and/or capable of being electronically erased and/or rewritten. In some aspects, the memory 416 may be, for example and without limitations a Flash memory.

In some aspects, the received sensor data may be for multiple instances of time. In some aspects, the received sensor data may include, for example and without limitation, light measurements and temperature measurements. In some aspects, the received sensor data may include time stamps, which may each indicate an instance of time at which the analyte sensor 102 took one or more of the light measurements and one or more of the temperature measurements. In some aspects, each time stamp may be a count of cycles of the clock 234. However, it is not required that received sensor data includes time stamps, and, in some aspects, the display device 106 may add time stamps to the received sensor data (e.g., if the display device 106 receives the sensor data as analyte sensor 102 generates the sensor data).

In some aspects, the processing circuitry 414 may use the sensor data to calculate analyte levels (e.g. glucose levels). In some aspects, the processing circuitry 414 may use the sensor data to calculate blood analyte levels. In some aspects in which the processing circuitry 414 calculates blood analyte levels, for each of the instances of time, the processing circuitry 414 may use the sensor data to calculate an ISF analyte level, may calculate a rate of change of the ISF analyte level, and may calculate a blood analyte level based on the calculated ISF glucose level and the calculated rate of change of the ISF analyte level. In some aspects, the processing circuitry 414 may store the calculated analyte levels in the memory 416.

In some aspects, the display device 106 may include a transceiver interface device. In some aspects, the transceiver interface device may include the second antenna 406 and the second wireless communication circuitry 408. In some aspects, the second wireless communication circuitry 408 may enable wireless communication by the display device 106 with one or more external devices, such as, for example, one or more personal computers, one or more transceivers 104, and/or one or more other display devices 106 via the second antenna 406. In some aspects, the second wireless communication circuitry 408 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some aspects, the second antenna 406 may be, for example and without limitation, a Bluetooth antenna.

In some aspects, the display device 106 may use the second antenna 406 and the second wireless communication circuitry 408 to receive sensor data and/or calculated analyte levels from the transceiver 104. In some aspects, the processing circuitry 414 may store the received sensor data and/or the received calculated analyte levels in the memory 416. In some aspects, the processing circuitry 414 may use the sensor data to calculate analyte levels (e.g., glucose levels). In some aspects (e.g., some aspects in which the display device 106 does not receive calculated analyte levels from transceiver 104), the processing circuitry 414 may calculate analyte levels based on the sensor data received from the transceiver 1204. In some aspects in which the processing circuitry 414 calculates blood analyte levels, for each of the instances of time, the processing circuitry 414 may use the sensor data to calculate an ISF analyte level, may calculate a rate of change of the ISF analyte level, and may calculate a blood analyte level based on the calculated ISF analyte level and the calculated rate of change of the ISF analyte level. In some aspects, the processing circuitry 414 may store the calculated analyte levels in the memory 416.

In some aspects in which the display device 106 includes the third antenna 410 and the third wireless communication circuitry 412, the third antenna 410 and the third wireless communication circuitry 412 may enable the display device 106 to communicate with one or more remote devices (e.g., smartphones, servers, and/or personal computers) via wireless local area networks (e.g., Wi-Fi), cellular networks, and/or the Internet. In some aspects, the third wireless communication circuitry 412 may employ one or more wireless communication standards to wirelessly transmit data. In some aspects, the third antenna 410 may be, for example and without limitation, a Wi-Fi antenna and/or one or more cellular antennas.

In some aspects in which the display device 106 includes the user interface 418, the user interface 418 may include a display 422 and/or a user input 420. In some aspects, the display 422 may be a liquid crystal display (LCD) and/or light emitting diode (LED) display. In some aspects, the user input 420 may include one or more buttons, a keyboard, a keypad, and/or a touchscreen. In some aspects, the processing circuitry 414 may control the display 422 to display data (e.g., predicted blood analyte levels, blood analyte trend information, alerts, alarms, and/or notifications). In some aspects, the user interface 418 may include one or more of a speaker 424 (e.g., a beeper) and a vibration motor, which may be activated, for example, in the event that a condition (e.g., a hypoglycemic or hyperglycemic condition) is met.

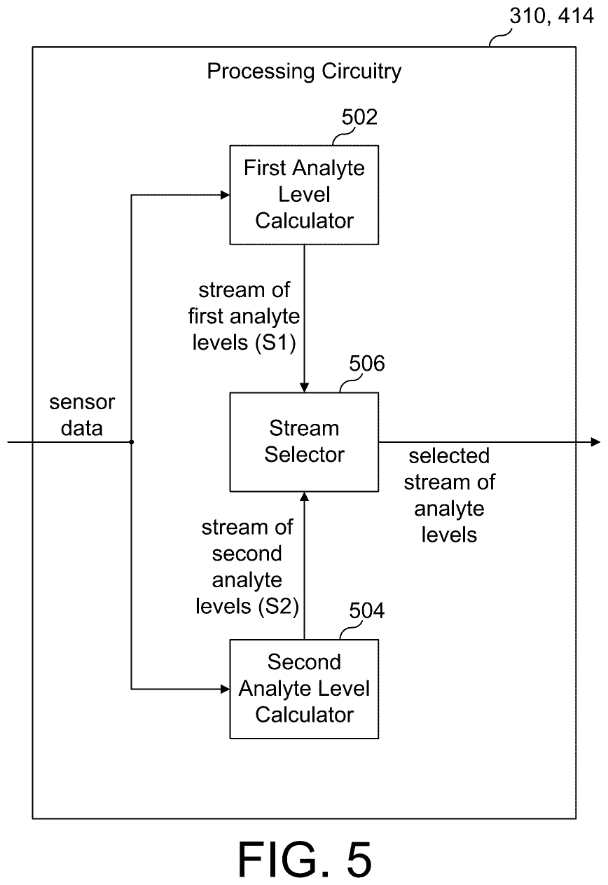

FIG. 5 is a block diagram illustrating an aspect of processing circuitry 310 of the transceiver 104 or processing circuitry 414 of the display device 106 according to some aspects. As shown in FIG. 5, in some aspects, the processing circuitry 310 or 414 may calculate analyte levels based on sensor data received, directly or indirectly, from the analyte sensor 102. In some aspects, the processing circuitry 310 or 414 may include a first analyte level calculator 502, a second analyte level calculator 504, and a stream selector 506. In some aspects, the processing circuitry 310 or 414 may receive sensor data for multiple instances of time, and the sensor data may have been conveyed by the analyte sensor 102. In some aspects, the first analyte level calculator 502 may be configured to use a first method to calculate a stream S1 of first analyte levels for the multiple instances of time based on at least the sensor data for the multiple instances of time. In some aspects, the second analyte level calculator 504 may be configured to use a second method to calculate a stream S2 of second analyte levels for the multiple instances of time based on at least the sensor data for the multiple instances of time. In some aspects, the second calculation method may be different than the first calculation method. In some aspects, the stream selector 506 may be configured to select, for each of the multiple instances of time, one of the stream S1 of first analyte levels and the stream S2 of second analyte levels. In some aspects, the analyte monitoring system 100 may be configured to, for each of the multiple instances of time, use the analyte level of the selected stream as the analyte level determined by the analyte monitoring system 100 for that instance of time. In some aspects in which the analyte monitoring system 100 is a continuous glucose monitoring (CGM) system, the determined analyte level may be a CGM level. In some aspects, the analyte monitoring system 100 may be configured to use analyte levels of the selected stream for display (e.g., by the display 422 of the user interface 418 of the display device 106).

In some aspects, the first method used by the first analyte level calculator 502 to calculate the stream S1 of first analyte levels may compensate for dynamic changes in opacity levels of the indicator 204 (e.g., hydrogel) containing the analyte indicator molecules 206 and the interferent indicator molecules 208. In some aspects, the first method may use measurements obtained from the reference photodetector 218, which may capture the first excitation light emitted by the first light source 212 and reflected back from the indicator 204, to infer opacity levels of the indicator 203 and compensate for the inferred opacity levels to improve for the accuracy of the calculated analyte levels. In some aspects, the first method may be a ratio method. In some aspects in which the analyte is glucose, the ratio method may calculate an interstitial fluid (ISF) glucose level as:

$$ISF Glucose = Kd ( T ) \frac{( {S}_{n} - 1 )}{( {S}_{nmax} ( T ) - {S}_{n} )} , \qquad ( 1 )$$

where ISF glucose is the ISF glucose level, T is the temperature (e.g., as measured by the temperature transducer 226), Kd(T) is the association-dissociation energy between glucose and the analyte indicator molecules 206, Sn is the normalized first emission light from the analyte indicator molecules 206 (e.g., where the raw optical signal measured by the one or more signal photodetectors 216 includes three components: I+Z+I_{distortion}, I is the first emission light from the analyte indicator molecules 206, Z is the total spillover from the first light source 212, I_{distortion} is a distortion of the analyte indicator molecules 206, and Sn=I/I₀, where I₀ is the baseline emission light from the analyte indicator molecules 206 at zero analyte concentration), Snmax(T) is the normalized fluorescence of a fully bound analyte indicator molecule 206 as a function of temperature T,

$$Kd ( T ) = {e}^{( {Kd}_{2} + {Kd}_{1} / ( 237.15 + T ) )} \qquad ( 2 )$$

$${S}_{nmax} ( T ) = {S}_{nmax} ( 37 ) * \frac{1 - {c}_{fmax} * ( T - 37 )}{1 - {c}_{f} * ( T - 37 )} , \qquad ( 3 )$$

$${S}_{n} = \frac{( {S}_{{n}_{1} WRef} + β * {S}_{{n}_{2} WRef} )}{RatioOpacityMF 1 * β * {S}_{{n}_{4} WRef}} + {S}_{{n}_{3} WRef} \qquad ( 4 )$$

$${S}_{{n}_{1} WRef} = \frac{Signal - {Z}_{bleed} / [ 1 + {c}_{Z} * ( T - 37 ) ]}{{e}^{- {k}_{ox} {t}_{ox}} * {e}^{- {k}_{th} {t}_{th}} * {e}^{- {k}_{pb} {t}_{pb}} * [ 1 - {c}_{f} * ( T - 37 ) ]} \qquad ( 5 )$$

$${S}_{{n}_{2} WRef} = \frac{Reference - {Ref\_Z}_{bleed} / [ 1 + {Ref\_c}_{Z} * ( T - 37 ) ]}{{e}^{- {k}_{ox} {t}_{ox}} * {e}^{- {k}_{th} {t}_{th}} * {e}^{- {Ref\_k}_{pb} {t}_{pb}} * [ 1 - {c}_{f} * ( T - 37 ) ]} \qquad ( 6 )$$

$${S}_{{n}_{3} WRef} = - \frac{{I}_{distortion , i n - vitro}}{{I}_{0 , i n - vitro} * {e}^{- {k}_{ox} {t}_{ox}} * {e}^{- {k}_{th} {t}_{th}} * {e}^{- {k}_{pb} {t}_{pb}} * [ 1 - ( T - 37 ) * {c}_{f} ]} \qquad ( 7 )$$

$${S}_{{n}_{4} WRef} = \frac{( Reference - {Ref\_Z}_{bleed} / [ 1 + {Ref\_c}_{z} * ( T - 37 ) ] ) * [ 1 + {T}_{{coeff}_{1}} * ( T - 37 ) + {T}_{{coeff}_{2}} * {( T - 37 )}^{2} ]}{1 + Ref\_ϕ * ( 1 - {e}^{- {Ref\_k}_{pb} {t}_{pb}} ) ]} , \qquad ( 8 )$$

I₀ is a baseline fluorescence at zero glucose, Kd1 and Kd2 are values obtained from quality control for temperature correction, C_{f} and C_{fmax} represent the temperature correction factor for the analyte indicator molecule 206 without glucose and fully bound, RatioOpacityMF1 is a calibration parameter that relates the opacity of the indicator 203 at 37° C. to the baseline fluorescence at zero glucose concentration value at 37° C., β is a constant from quality control, Z_{bleed} represents light from spillover of the first light source 212, c_{z} and c_{f} are temperature correction coefficients, Tcoeff1 and Tcoeff2 are coefficients for temperature correction on the reference, φ is quantum efficiency, and I_{distortion,in-vitro} represents non-glucose-modulated fluorescent light emitted from oxidized, thermally degraded, and photo-activated indicator species.

In some aspects, after calculating the ISF glucose level using the first method, the first analyte level calculator 502 may perform lag compensation to convert the ISF glucose level into a blood glucose level for the stream S1 of first analyte levels. In some aspects, the first analyte level calculator 502 may use a two-compartment model for lag compensation. In some aspects, the first analyte level calculator 502 may calculate the blood glucose level using at least the calculated ISF glucose level and a calculated rate of change of the ISF glucose level (ISF_ROC). In some aspects, the first analyte level calculator 502 may calculate the blood glucose level as ISF_ROC/p2+(1+p3/p2)*ISF_glucose, where p2 is the analyte diffusion rate, p3 is the analyte consumption rate, and ISF_glucose is the calculated ISF glucose level.

In some aspects, the second method used by the second analyte level calculator 504 to calculate the stream S2 of second analyte levels may calibrate both gain and offset parameters of the analyte sensor 102 in real time. In some aspects, the second method may be a two-parameter method. In some aspects in which the analyte is glucose, like the ratio method, the two-parameter method may calculate an interstitial fluid (ISF) glucose level as:

$$ISF Glucose = Kd ( T ) \frac{( {S}_{n} - 1 )}{( {S}_{nmax} ( T ) - {S}_{n} )} , \qquad ( 9 )$$

where ISF glucose is the ISF glucose level, T is the temperature (e.g., as measured by the temperature transducer 226), Kd(T) is the association-dissociation energy between glucose and the analyte indicator molecules 206, Sn is the normalized first emission light from the analyte indicator molecules 206, Snmax(T) is the normalized fluorescence of a fully bound analyte indicator molecule 206 as a function of temperature T, Snmax(T)=Snmax₁*T+Snmax₂, Snmax₁ and Snmax₂ are coefficients of temperature dependence,

$$Kd ( T ) = {e}^{( {Kd}_{2} + {Kd}_{1} / ( 237.15 + T ) )} \qquad ( 10 )$$

$${S}_{n} = \frac{1}{Gain} * {S}_{n 1} + \frac{Offset}{Gain} * {S}_{n 2} + {S}_{n 3} \qquad ( 11 )$$

$${S}_{n 1} = \frac{Signal * [ 1 + {c}_{Z} * ( T - 37 ) ] - {Z}_{bleed}}{{I}_{0 , i n - vitro} * {e}^{- {k}_{ox} {t}_{ox}} * {e}^{- {k}_{th} {t}_{th}} * {e}^{- {k}_{pb} {t}_{pb}} * [ 1 - ( T - 37 ) * {c}_{f} ]} , \qquad ( 12 )$$

$${S}_{n 2} = - \frac{{Z}_{gel , i n - vitro} * ( ( T - 37 ) * {e}^{- {k}_{pb} {t}_{pb}} * {c}_{ab s} + 1 ) * [ 1 + {φ}_{Z} * ( 1 - {e}^{- {k}_{pb} {t}_{pb}} ) ]}{{I}_{0 , i n - vitro} * {e}^{- {k}_{ox} {t}_{ox}} * {e}^{- {k}_{th} {t}_{th}} * {e}^{- {k}_{pb} {t}_{pb}} * [ 1 - ( T - 37 ) * {c}_{f} ]} , \qquad ( 13 )$$

$${S}_{n 3} = - \frac{{I}_{distortion , i n - vitro}}{{I}_{0 , i n - vitro} * {e}^{- {k}_{ox} {t}_{ox}} * {e}^{- {k}_{th} {t}_{th}} * {e}^{- {k}_{pb} {t}_{pb}} * [ 1 - ( T - 37 ) * {c}_{f} ]} , \qquad ( 14 )$$

I₀ is a baseline fluorescence at zero glucose, Kd1 and Kd2 are values obtained from quality control for temperature correction, C_{f} represents the temperature correction factor for the analyte indicator molecule 206 without glucose and fully bound, Z_{bleed} represents light from spillover of the first light source 212, I_{distortion, in-vitro} represents non-glucose-modulated fluorescent light emitted from oxidized, thermally degraded, and photo-activated indicator species, and Gain and Offset are calibration parameters that describe changes in the modulatable and non-modulatable portions of the signal, respectively.

In some aspects, after calculating the ISF glucose level using the second method, the second analyte level calculator 504 may perform lag compensation to convert the ISF glucose level into a blood glucose level for the stream S2 of second analyte levels. In some aspects, the second analyte level calculator 504 may use the two-compartment model for lag compensation. In some aspects, the second analyte level calculator 504 may calculate the blood glucose level using at least the calculated ISF glucose level and a calculated rate of change of the ISF glucose level (ISF_ROC). In some aspects, the second analyte level calculator 504 may calculate the blood glucose level as ISF_ROC/p₂+(1+p₃/p₂)*ISF_glucose, where p₂ is the analyte diffusion rate, p₃ is the analyte consumption rate, and ISF_glucose is the calculated ISF glucose level.

Although in some aspects, the first method may be the ratio method, and the second method may be the two-parameter method, this is not required. For example, in some alternative aspects, the first method may be the two-parameter method, and the second method may be the ratio method. In some further alternative aspects, one of the first and second methods may be one of the ratio and two-parameter methods, and the other of the first and second methods may be a calculation method other than the ratio and two-parameter methods. In some additional alternative aspects, both of the first and second methods may be calculation methods other than the ratio and two-parameter methods. In some aspects, the first method may be a known analyte method calculation method, and the second method may be a different known analyte method calculation method.

In some aspects, the first and second analyte level calculators 502 and 504 may calculate the analyte levels of the first and second streams S1 and S2 in parallel. In some aspects, the stream selector 506 may treat the first and second streams S1 and S2 as primary and secondary streams, respectively. In some aspects, the stream selector 506 may select the stream S1 of first analyte levels by default, and, thus, the analyte monitoring system 100 may use the stream S1 of first analyte levels for display by default. In some aspects, the stream selector 506 may be configured to determine whether to switch the analyte monitoring system 100 from using the stream S1 of first analyte levels for display to using the stream S2 of second analyte levels for display. In some aspects, the stream selector 506, in determining whether to switch from the stream S1 of first analyte levels to the stream S2 of second analyte levels for display, may be configured to compare one or more of the first analyte levels to one or more of the second analyte levels.

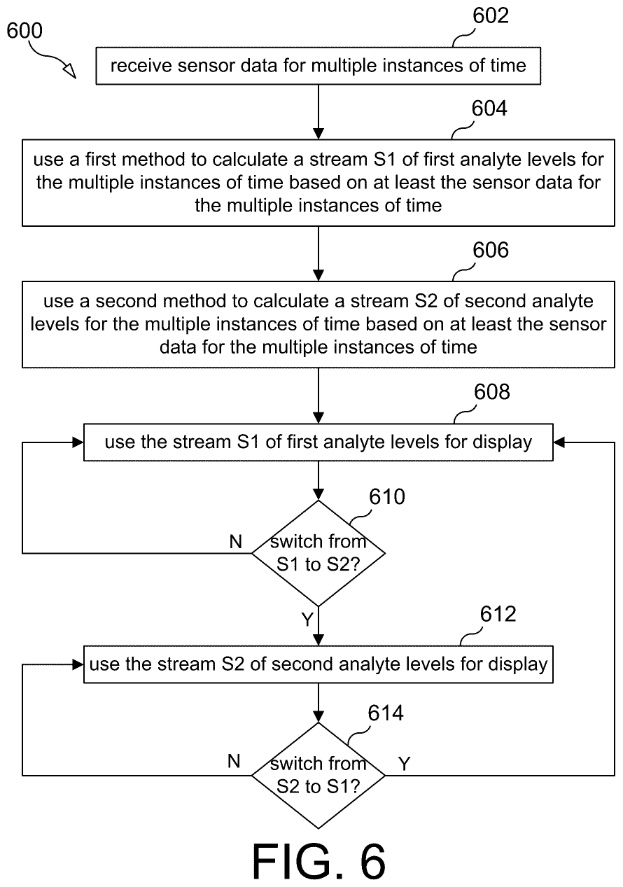

FIG. 6 illustrates a process 600 according to some aspects. In some aspects, one or more steps of the process 600 may be performed by the processing circuitry 310 of the transceiver 104. In some alternative aspects, one or more steps of the process 600 may be performed by the processing circuitry 414 of the display device 106.

In some aspects, as shown in FIG. 6, the process 600 may include a step 602 of receiving sensor data for multiple instances of time. In some aspects, the sensor data was conveyed, directly or indirectly, by the analyte sensor 102. In some aspects in which the processing circuitry 310 of the transceiver 104 performs one or more steps of the process 600, the processing circuitry 310 of the transceiver 104 may receive the sensor data directly from the analyte sensor 102 using the first wireless communication circuitry 304 and first antenna 302 of the transceiver 104. In some alternative aspects in which the processing circuitry 414 of the display device 106 performs one or more steps of the process 600, the processing circuitry 414 of the display device 106 may receive the sensor data directly from the analyte sensor 102 using the first wireless communication circuitry 404 and first antenna 402 of the display device 106 and/or indirectly from the analyte sensor 102 via the transceiver 104 using the second wireless communication circuitry 408 and second antenna 406 of the display device 106. In some aspects, the sensor data for the multiple instances of time may be received together, and/or the sensor data for the multiple instances of time may be received separately (e.g., for each instance of time).

In some aspects, as shown in FIG. 6, the process 600 may include a step 604 in which the processing circuitry 310 or 414 (e.g., the first analyte level calculator 502 of the processing circuitry 310 or 410) uses a first method to calculate the stream S1 of first analyte levels for the multiple instances of time based on at least the sensor data for the multiple instances of time.

In some aspects, as shown in FIG. 6, the process 600 may include a step 606 in which the processing circuitry 310 or 414 (e.g., the second analyte level calculator 504 of the processing circuitry 310 or 410) uses a second method to calculate the stream S2 of second analyte levels for the multiple instances of time based on at least the sensor data for the multiple instances of time. In some aspects, the first method may be one of the ratio method and the two-parameter method, and the second method may be another of the ratio method and the two-parameter method. In some alternative aspects, other analyte level calculation methods may be used for one or more of the first and second methods.

In some aspects, as shown in FIG. 6, the process 600 may include a step 608 of using the stream S1 of first analyte levels for display. In some aspects, the step 608 may include the processing circuitry 310 or 414 (e.g., the stream selector 506 of the processing circuitry 310 or 414) selecting the stream S1 of the first analyte levels. In some aspects in which the processing circuitry 310 of the transceiver 104 performs one or more steps of the process 600, the step 608 may include the processing circuitry 310 using the second wireless communication circuitry 308 and the second antenna 306 to convey first analyte levels of the stream S1 to the display device 106, which may receive and display the first analyte levels (e.g., using the display 422 of the user interface 418). In some aspects in which the processing circuitry 414 of the display device 106 performs one or more steps of the process 600, the step 608 may include the processing circuitry 414 displaying first analyte levels of the stream S1 (e.g., using the display 422 of the user interface 418).

In some aspects, as shown in FIG. 6, the process 600 may include a step 610 in which the processing circuitry 310 or 414 (e.g., the stream selector 506 of the processing circuitry 310 or 414) determines whether to switch from use of the stream S1 of first analyte levels for display to use of the stream S2 of second analyte levels for display. In some aspects, determining whether to switch from use of the stream S1 of first analyte levels for display to use of the stream S2 of second analyte levels in step 610 may include comparing one or more of the first analyte levels to one or more of the second analyte levels. In some aspects, the process 600 may return to the step 608 and continue using the stream S1 of first analyte levels for display if the processing circuitry 310 or 414 (e.g., the stream selector 506 of the processing circuitry 310 or 414) determines in step 610 to not switch from use of the stream S1 of first analyte levels to use of the stream S2 of second analyte levels.

In some aspects, as shown in FIG. 6, the process 600 may include a step 612 of switching to use of the stream S2 of second analyte levels for display if the processing circuitry 310 or 414 (e.g., the stream selector 506 of the processing circuitry 310 or 414) determines in step 610 to switch from use of the stream S1 of first analyte levels to use of the stream S2 of second analyte levels. In some aspects, the step 612 may include the stream selector 506 of the processing circuitry 310 or 414 selecting the stream S2 of the second analyte levels. In some aspects in which the processing circuitry 310 of the transceiver 104 performs one or more steps of the process 600, the step 612 may include the processing circuitry 310 using the second wireless communication circuitry 308 and the second antenna 306 to convey second analyte levels of the stream S2 to the display device 106, which may receive the second analyte levels (e.g., using the second wireless communication circuitry 408 and the second antenna 406) and display the second analyte levels (e.g., using the display 422 of the user interface 418). In some aspects in which the processing circuitry 414 of the display device 106 performs one or more steps of the process 600, the step 612 may include the processing circuitry 414 using the display 422 of the user interface 418 to display second analyte levels of the stream S2.

In some aspects, as shown in FIG. 6, the process 600 may include a step 614 in which the stream selector 506 the processing circuitry 310 or 414 (e.g., the stream selector 506 of the processing circuitry 310 or 414) determines whether to switch from use of the stream S2 of second analyte levels for display to use of the stream S1 of first analyte levels for display. In some aspects, the process 600 may return to the step 612 and continue using the stream S2 of second analyte levels for display if the processing circuitry 310 or 414 (e.g., the stream selector 506 of the processing circuitry 310 or 414) determines in step 614 to not switch from use of the stream S2 of second analyte levels to use of the stream S1 of first analyte levels. In some aspects, the process 600 may return to the step 608 and switch to use of the stream S1 of first analyte levels for display if the processing circuitry 310 or 414 (e.g., the stream selector 506 of the processing circuitry 310 or 414) determines in step 614 to switch from use of the stream S2 of second analyte levels to use of the stream S1 of first analyte levels.

In some aspects, the dynamic stream selection (DSS) of process 600 (e.g., in step 610 and/or step 614) may improve the accuracy of the analyte levels selected for display by the analyte monitoring system 100 (e.g., when compared to venous blood analyte measurements). In some aspects, the stream having lower analyte levels may generally be the more accurate stream (e.g., when compared to venous blood analyte measurements). This may be especially true for analyte levels below a first analyte level threshold, which may be, for example and without limitation, 100 mg/dL or 110 mg/dL. In some aspects, the stream having lower analyte levels may allow increased sensitivity to detecting hypoglycemic events. In some aspects, the DSS of process 600 may improve the accuracy of the analyte levels selected for display by the analyte monitoring system 100 generally switching to the stream of analyte levels having lower analyte levels.

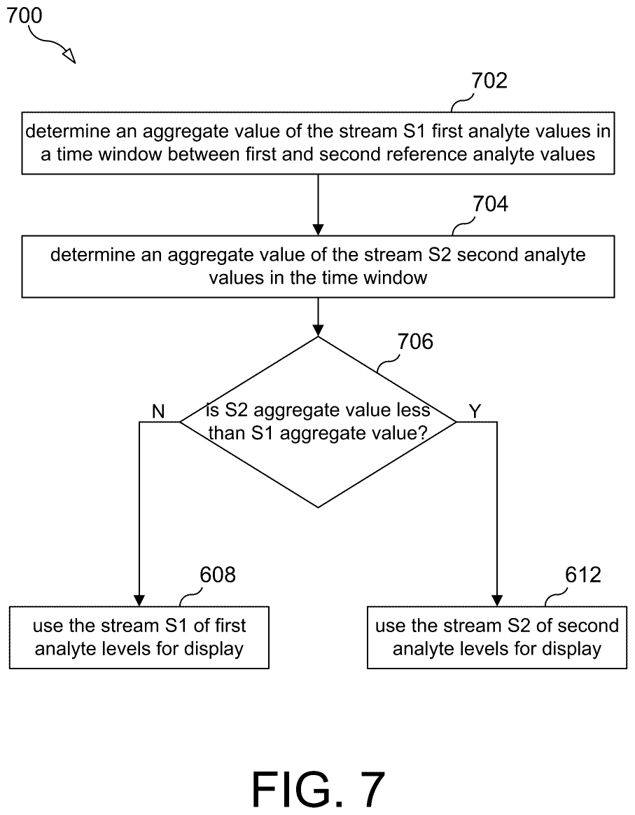

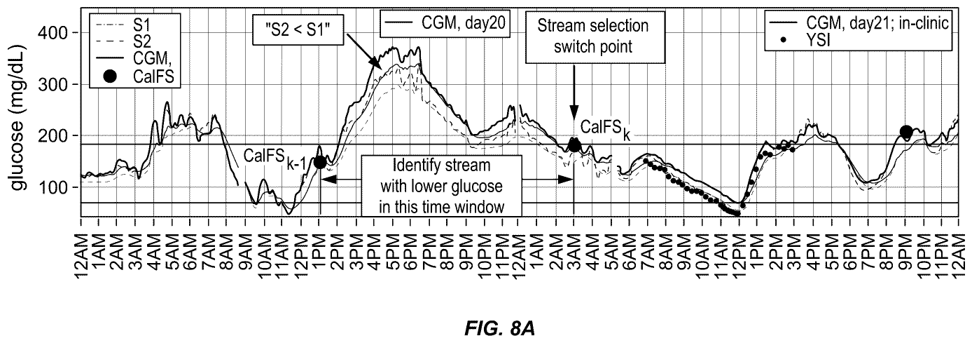

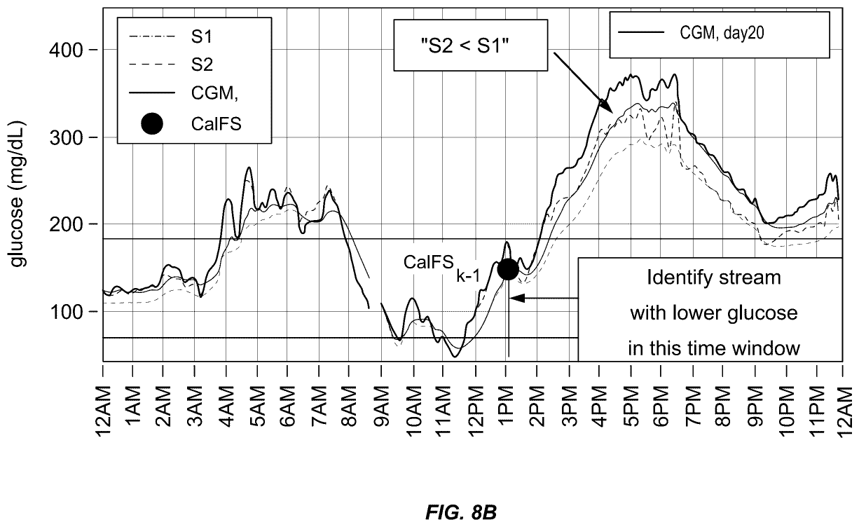

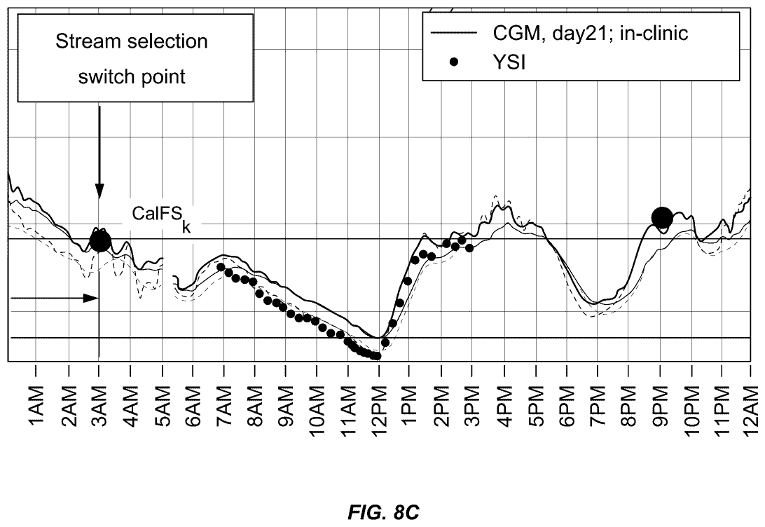

FIG. 7 illustrates a first dynamic stream selection (DSS1) process 700 according to some aspects. In some aspects, the DSS1 process 700 may be performed in step 610 and/or step 614 of the process 600 shown in FIG. 6. In some aspects, the process 700 may include comparing one or more of the first analyte levels of the stream S1 to one or more of the second analyte levels of the stream S2. In some aspects, one or more steps of the DSS1 process 700 may be performed by the processing circuitry 310 of the transceiver 104. In some alternative aspects, one or more steps of the DSS1 process 700 may be performed by the processing circuitry 414 of the display device 106. FIGS. 8A-8C illustrate a glucose monitoring example of the DSS1 process 700. FIGS. 8B and 8C show enlarged left and right halves, respectively, of the graph shown in FIG. 8A. In FIGS. 8A-8C, venous blood glucose measurements are labeled as YSI measurements, and the analyte levels calculated by the analyte monitoring system 100 for display are compared to the venous glucose measurements when assessing the accuracy of the analyte monitoring system 100. As shown in FIGS. 8A and 8C, selecting stream 2 at 3 AM of day 21 results in display of more accurate glucose levels calculated by the analyte monitoring system 100 (when compared to the venous blood analyte measurements) than if stream 1 were selected.

In some aspects, the process 600 described above with respect to FIG. 6 may include steps in which the analyte monitoring system 100 receives reference analyte values for respective reference instances of time. In some aspects, the analyte monitoring system 100 may receive the reference analyte values via the use input 420 of the use interface 418 of the display device 106. In some aspects in which the stream selector 506 is part of the processing circuitry 310 of the transceiver 104, the processing circuitry 414 of the display device 106 may use the second wireless communication circuitry 408 and the second antenna 406 to convey received reference analyte values to the transceiver 104, which may be received by the second wireless communication circuitry 308 and the second antenna 306 of the transceiver 104. In some aspects in which the stream selector 506 is part of the processing circuitry 414 of the display device 106, the processing circuitry 414 of the display device 106 may receive the reference analyte values from the user interface 418. In some aspects, the reference analyte values may be finger stick calibration analyte measurements (CalFS), which are capillary blood analyte measurements.

In some aspects, the analyte values received by the analyte monitoring system 100 may include a first reference analyte value (e.g., CalFSk−1 in FIGS. 8A-8C) for a first reference instance of time (e.g., 1 PM of day 20 in FIGS. 8A-8C) and a second reference analyte value (e.g., CalFSk in FIGS. 8A-8C) for a second reference instance of time (e.g., 3 AM of day 21 in FIGS. 8A-8C). In some aspects, the processing circuitry 310 or 414 (e.g., the stream selector 506 of the processing circuitry 310 or 414) may be configured to determine whether to switch from use of the stream S1 of first analyte levels for display to use of the stream S2 of second analyte levels for display in step 610 if the analyte monitoring system 100 receives the second reference analyte value (CalFSk) for the second reference instance of time.

In some aspects, as shown in FIG. 7, the DSS1 process 700 may include a step 702 of determining an aggregate value of the first analyte levels that are for instances of the multiple instances of time that are in the time window between the first and second reference instances of time (e.g., an aggregate value of the first analyte levels of the stream S1 that are for instances of time between 1 PM of day 20 and 3 AM of day 21 in FIGS. 8A-8C). In some aspects, the determined aggregate value of the first analyte levels in the time window may be, for example and without limitation, a first quartile of the first analyte levels that are in the time window. In some aspects, the first quartile of the first analyte levels may be an analyte level that is (1) above the lowest 25% of the first analyte levels in the time window and (2) below the highest 75% of the first analyte levels in the time window. In some alternative aspects, the aggregate value may instead be (a) an average value of the first analyte levels in the time window, (b) a median value of the first analyte levels in the time window, or (c) an analyte value above a different percentage (e.g., 15%, 20%, 30%, or 35%) of the first analyte levels in the time window.

In some aspects, as shown in FIG. 7, the DSS1 process 700 may include a step 704 of determining an aggregate value of the second analyte levels that are for instances of the multiple instances of time that are in the time window between the first and second reference instances of time. In some aspects, the determined aggregate value of the second analyte levels in the time window may be a first quartile of the second analyte levels in the time window. In some alternative aspects, the aggregate value may instead be (a) an average value of the second analyte levels in the time window, (b) a median value of the second analyte levels in the time window, or (c) an analyte value above a different percentage (e.g., 15%, 20%, 30%, or 35%) of the second analyte levels in the time window.

In some aspects, the DSS1 process 700 may include a step 706 of determining whether the aggregate value of the second analyte levels is lower than the aggregate value of the first analyte levels. In some aspects, if the DSS1 process 700 is being performed in step 610 of the process 600 shown in FIG. 6, the DSS1 process 700 may determine to not switch from the use of the stream S1 of first analyte levels for display to use of the stream S2 of second analyte levels for display if the aggregate value of the second analyte levels is not determined to be lower than the aggregate value of the first analyte levels, and the process 600 may return to step 608. In some aspects, if the DSS1 process 700 is being performed in step 610 of the process 600 shown in FIG. 6, the DSS1 process 700 may determine to switch from the use of the stream S1 of first analyte levels for display to use of the stream S2 of second analyte levels for display if the aggregate value of the second analyte levels is determined to be lower than the aggregate value of the first analyte levels, and the process 600 may proceed to step 612. In some aspects, if the DSS1 process 700 is being performed in step 614 of the process 600 shown in FIG. 6, the DSS1 process 700 may determine to switch from the use of the stream S2 of second analyte levels for display to use of the stream S1 of first analyte levels for display if the aggregate value of the second analyte levels is not determined to be lower than the aggregate value of the first analyte levels, and the process 600 may proceed to step 608. In some aspects, if the DSS1 process 700 is being performed in step 614 of the process 600 shown in FIG. 6, the DSS1 process 700 may determine to not switch from the use of the stream S2 of second analyte levels for display to use of the stream S1 of first analyte levels for display if the aggregate value of the second analyte levels is determined to be lower than the aggregate value of the first analyte levels, and the process 600 may return to step 612.

In some aspects, the received reference analyte values may include a third reference analyte value for a third reference instance of time, which is subsequent to the second reference instance of time. In some aspects, the DSS1 process 700 may include a step of determining a second aggregate value of the first analyte levels that are for instances of the multiple instances of time that are in a second time window between the second and third reference instances of time. In some aspects, the DSS1 process 700 may include a step of determining a second aggregate value of the second analyte levels in the second window of time. In some aspects, the DSS1 process 700 may include a step of determining whether the second aggregate value of the second analyte levels is lower than the second aggregate value of the first analyte levels. In some aspects, the DSS1 process 700 may include using the stream of first analyte levels for display for one or more instances of the multiple instances of time that are subsequent to the third reference instances of time if the second aggregate value of the second analyte levels is not determined to be lower than the second aggregate value of the first analyte levels, and using the stream of second analyte levels for display for the one or more instances of the multiple instances of time that are subsequent to the third reference instances of time if the second aggregate value of the second analyte levels is determined to be lower than the second aggregate value of the first analyte levels.

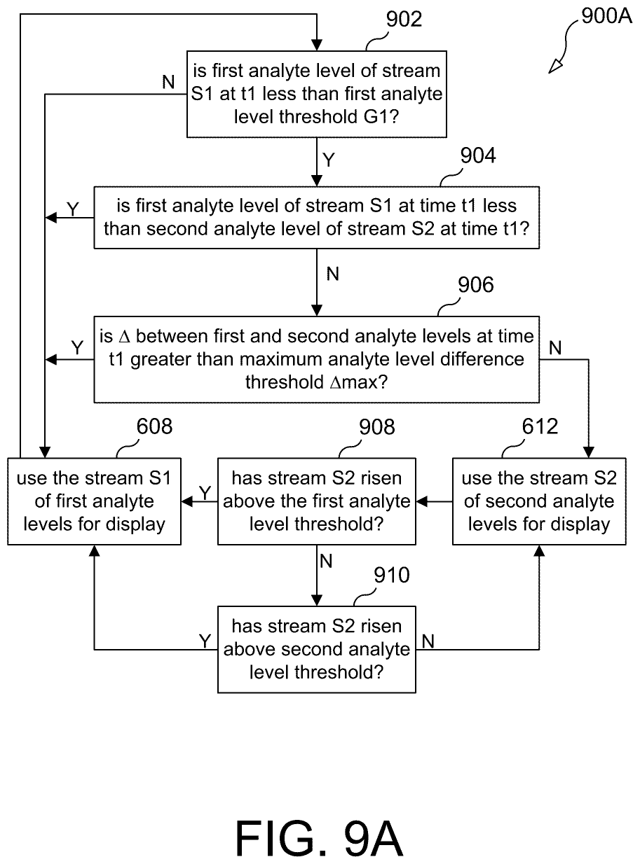

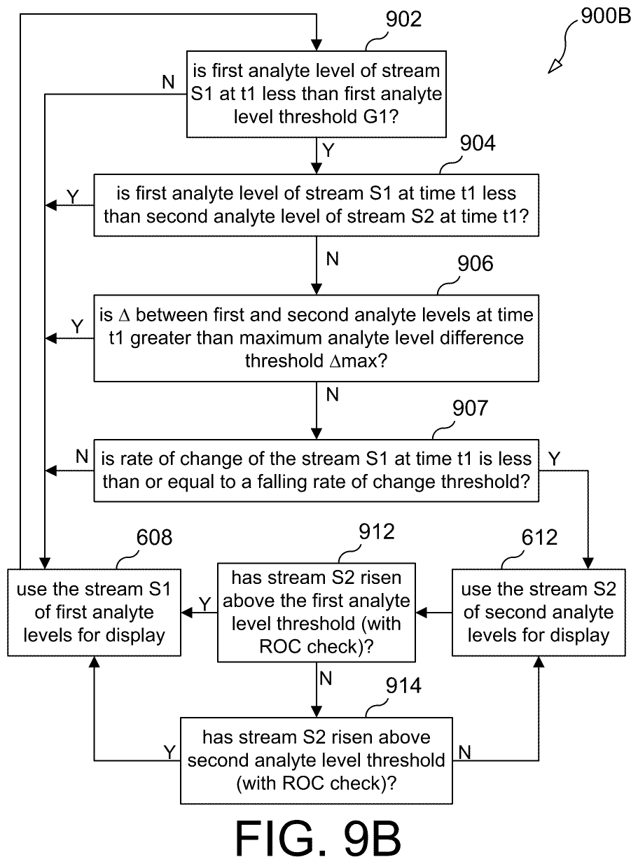

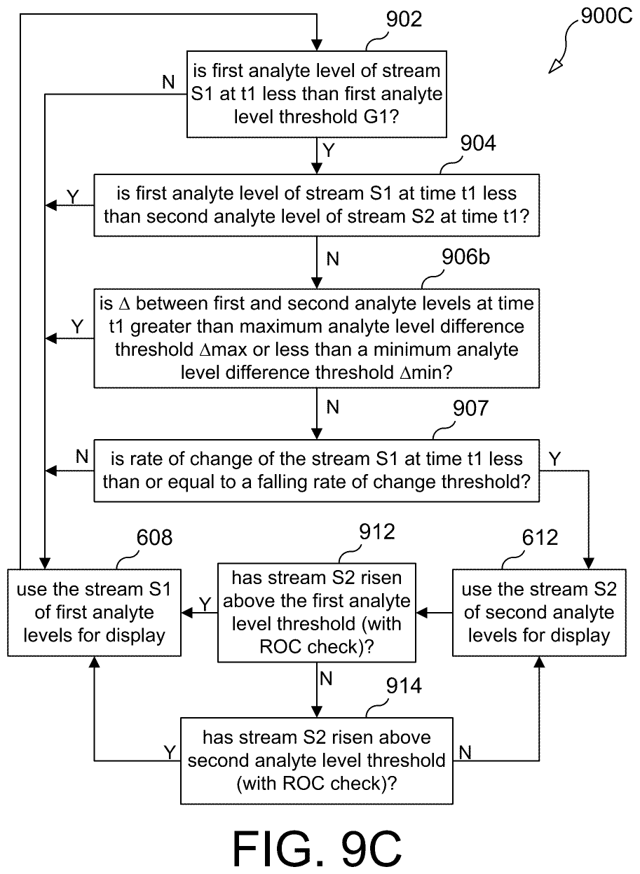

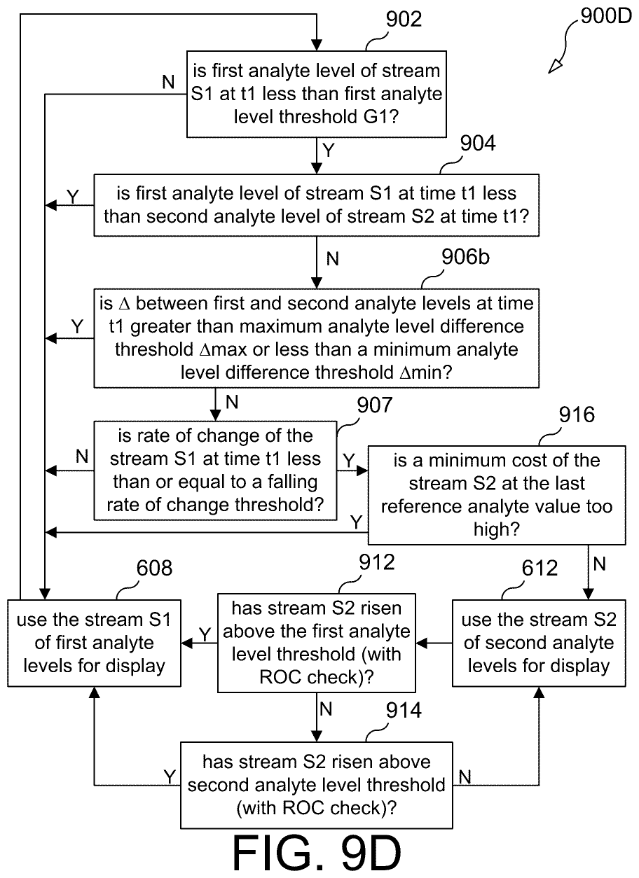

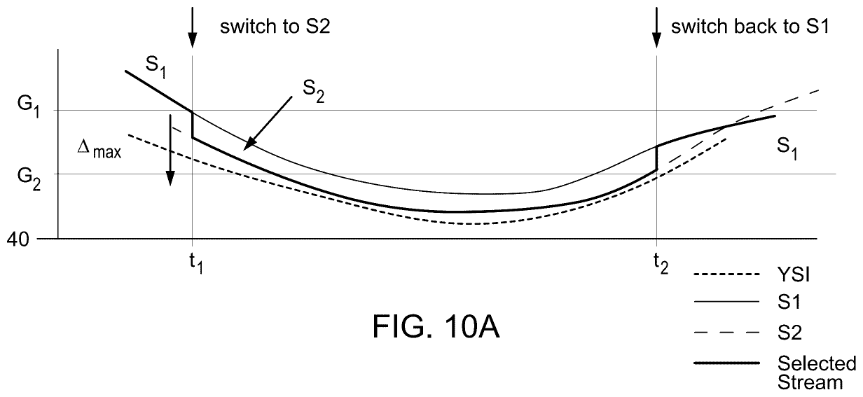

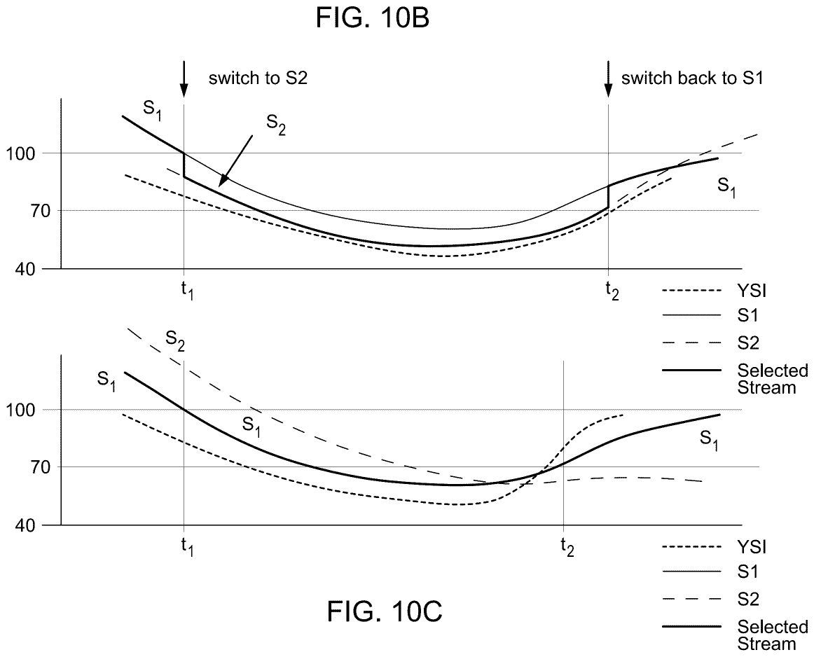

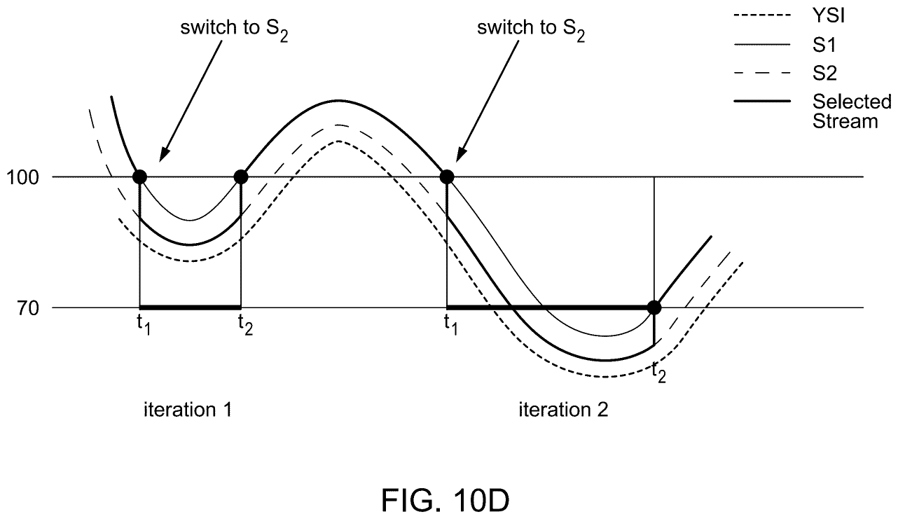

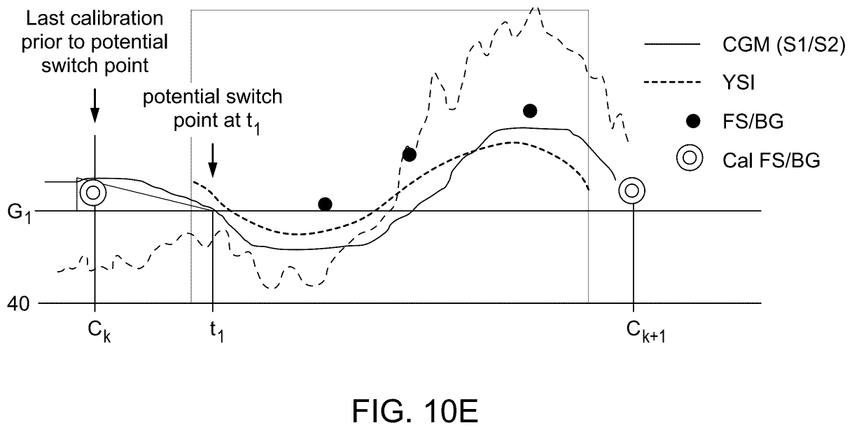

FIG. 9A illustrates a second dynamic stream selection (DSS2) process 900A according to some aspects. In some aspects, the DSS2 process 900A may be performed in step 610 and/or step 614 of the process 600 shown in FIG. 6. In some aspects, the process 900A may include comparing one or more of the first analyte levels of the stream S1 to one or more of the second analyte levels of the stream S2. In some aspects, one or more steps of the DSS2 process 900A may be performed by the processing circuitry 310 of the transceiver 104. In some alternative aspects, one or more steps of the DSS2 process 900A may be performed by the processing circuitry 414 of the display device 106. FIGS. 10A-10D illustrate examples of the DSS2 process 900A. In FIGS. 10A-10D, the stream S1 is shown with a solid line when the stream S1 is not the selected stream of analyte values, and the stream S2 is shown with a long-dashed line when the stream S2 is not the selected stream of analyte value. In FIGS. 10A-10D, the selected stream of analyte values is shown as a bolded line. In FIGS. 10A-10D, venous blood analyte measurements are labeled as YSI measurements and shown with a bolded, short-dashed line, and the analyte levels calculated by the analyte monitoring system 100 for display are compared to the venous analyte measurements when assessing the accuracy of the analyte monitoring system 100. In the examples of FIGS. 10A, 10B, and 10D, selecting stream 2 at time t₁ results in display of more accurate analyte levels calculated by the analyte monitoring system 100 (when compared to the venous blood analyte measurements) than if stream 1 were selected. In contrast, in the example of FIG. 10C, selecting stream 1 at time t₁ results in display of more accurate analyte levels calculated by the analyte monitoring system 100 (when compared to the venous blood analyte measurements) than if stream 2 were selected.

In some aspects, as shown in FIG. 9A, the DSS2 process 900A may include a step 902 in which the processing circuitry 310 or 414 (e.g., the stream selector 506 of the processing circuitry 310 or 414), in determining whether to switch from use of the stream S1 of first analyte levels for display to use of the stream S2 of second analyte levels in step 610 of the process 600, determines whether the first analyte level for a first instance (e.g., t₁ in the examples shown in FIGS. 10A-10D) of the multiple instances of time is less than a first analyte level threshold G1. In some alternative aspects, in step 902, the processing circuitry 310 or 414 (e.g., the stream selector 506 of the processing circuitry 310 or 414) may instead determine at the first instance of the multiple instances of time whether a predicted first analyte level is less than the first analyte level threshold G1. In some aspects, the predicted first analyte level may be the predicted first analyte level at a future time (e.g., 5 minutes, 10 minutes, or 15 minutes from the first instance of time). In some aspects, as shown in FIG. 10B-10D, the first analyte level threshold G1 may be 100 mg/dL. In some alternative aspects, the first analyte level threshold G1 may be a different value (e.g., 90 mg/dL, 95 mg/dL, 105 mg/dL, 110 mg/dL, 115 mg/dL, or 120 mg/dL). In some aspects, the maximum analyte level difference threshold may be 40 mg/dL.

In some aspects, as shown in FIG. 9A, the DSS2 process 900A may include a step 904 in which the processing circuitry 310 or 414 (e.g., the stream selector 506 of the processing circuitry 310 or 414), in comparing the one or more of the first analyte levels to the one or more of the second analyte levels in step 610 of the process 600, determines whether the second analyte level for the first instance (e.g., t1 in FIGS. 10A-10D) of the multiple instances of time is lower than the first analyte level for the first instance (e.g., t1 in FIGS. 10A-10D) of the multiple instances of time.

In some aspects, as shown in FIG. 9A, the DSS2 process 900A may include a step 906 in which the processing circuitry 310 or 414 (e.g., the stream selector 506 of the processing circuitry 310 or 414), in comparing the one or more of the first analyte levels to the one or more of the second analyte levels in step 610, determines whether a difference between the first and second analyte levels for the first instance of the multiple instances of time is greater than a maximum analyte level difference threshold Δmax.

In some aspects, as shown in FIG. 9A, in the DSS2 process 900A, determining whether to switch from use of the stream S1 of first analyte levels for display to use of the stream S2 of second analyte levels for display in step 610 of the process 600 may include determining to not switch from the use of the stream S1 of first analyte levels for display to use of the stream S2 of second analyte levels for display (and returning to step 608 in which the stream selector 506 continues to select and the analyte monitoring system 100 continues to use the stream S1 of first analyte levels for display) if (a) the first analyte level for the first instance of time is not less than the first analyte level threshold G1 (step 902), (b) the first analyte level for the first instance of time is less than the second analyte level for the first instance of time (step 904), and/or (c) the difference between the first and second analyte levels for the first instance of time is greater than the maximum analyte level difference threshold Δmax (step 906). For example, as shown in FIG. 10C, the stream S1 of first analyte values remains the selected stream and continues to be used for display after the first instance of time (t1) because the first analyte level for the first instance of time is less than the second analyte level for the first instance of time (t1).

In some aspects, as shown in FIG. 9A, in the DSS2 process 900A, determining whether to switch from use of the stream S1 of first analyte levels for display to use of the stream S2 of second analyte levels for display in step 610 of the process 600 may include determining to switch from the use of the stream S1 of first analyte levels for display to use of the stream S2 of second analyte levels for display (and proceeding to step 612 in which the stream selector 506 selects and the analyte monitoring system 100 uses the stream S2 of second analyte levels for display) if (i) the first analyte level for the first instance of time is less than the first analyte level threshold G1, (ii) the first analyte level for the first instance of time is greater than the second analyte level for the first instance of time, and (iii) the difference between the first and second analyte levels for the first instance of time is less than the analyte difference threshold Δmax. For example, as shown in FIGS. 10A, 10B, and 10D, the stream S2 of second analyte levels is selected at the first instance of time (t1), and the analyte monitoring system 100 switches from using of the stream S1 of first analyte levels for display to use of the stream S2 of second analyte levels for display at the first instance of time (t1) because conditions (i) through (iii) are met.

In some aspects, as shown in FIG. 9A, the DSS2 process 900A may include, subsequent to using of the stream S2 of second analyte levels for display in step 612 of the process 600, a step 908 in which the processing circuitry 310 or 414 (e.g., the stream selector 506 of the processing circuitry 310 or 414), in determining whether to switch from use of the stream S2 of second analyte levels for display to use of the stream S1 of first analyte levels for display in the step 614 of the process 600, determines whether the second analyte level for a second instance of the multiple instances of time is greater than the first analyte level threshold G1. In some aspects, as shown in FIG. 9A, the DSS2 process 900A may include, subsequent to switching to use of the stream S2 of second analyte levels for display by proceeding to step 612, switching to use of the stream S1 of first analyte levels for display by proceeding from step 908 to step 608 if the second analyte level for the second instance of the multiple instances of time is determined to be greater than the first analyte level threshold G1.

In some alternative aspects, the step 908 may instead include the processing circuitry 310 or 414 (e.g., the stream selector 506 of the processing circuitry 310 or 414), in determining whether to switch from use of the stream S2 of second analyte levels for display to use of the stream S1 of first analyte levels for display in the step 614 of the process 600, determining whether the first analyte level for the second instance of the multiple instances of time is greater than the first analyte level threshold G1. In these alternative aspects, the DSS2 process 900A may include, subsequent to switching to use of the stream S2 of second analyte levels for display by proceeding to step 612, switching to use of the stream S1 of first analyte levels for display by proceeding from step 908 to step 608 if the first analyte level for the second instance of the multiple instances of time is determined to be greater than the first analyte level threshold G1. For example, as shown in FIG. 10D, subsequent to switching to use of the stream S2 of second analyte levels for display at the first instance of time (t1 of iteration 1), the analyte monitoring system 100 switches to use of the stream S1 of first analyte levels for display at the second instance of time (t2 of iteration 1) because the first analyte level for the second instance of time (t2 of iteration 1) is determined to be greater than the first analyte level threshold G1, which is 100 mg/dL in FIG. 10D.

In some aspects, as shown in FIG. 9A, the DSS2 process 900A may include, subsequent to using of the stream S2 of second analyte levels for display in step 612 of the process 600, a step 910 in which the processing circuitry 310 or 414 (e.g., the stream selector 506 of the processing circuitry 310 or 414), in determining whether to switch from use of the stream S2 of second analyte levels for display to use of the stream S1 of first analyte levels for display in the step 614 of the process 600, (1) determines whether the second analyte level for a second instance of the multiple instances of time is less than a second analyte level threshold G2 and then (2) if the second analyte level for the second instance of the multiple instances of time is determined to be less than the second analyte level threshold, determining whether the second analyte level for a third instance of the multiple instances of time is greater than the second analyte level threshold. In some aspects, as shown in FIG. 9A, the DSS2 process 900A may include, subsequent to switching to use of the stream S2 of second analyte levels for display by proceeding to step 612, switching to use of the stream S1 of first analyte levels for display by proceeding from step 910 to step 608 if the second analyte level for the third instance of the multiple instances of time is determined to be greater than the second analyte level threshold G2. For example, as shown in FIGS. 10A and 10B, subsequent to switching to use of the stream S2 of second analyte levels for display at the first instance of time (t1), (1) the second analyte level for a second instance of time (a time between t1 and t2) is determined to be less than the second analyte level threshold G2, which is 70 mg/dL in FIG. 10B, and then (2) the analyte monitoring system 100 switches to use of the stream S1 of first analyte levels for display at the third instance of time (t2) because the second analyte level for the third instance of time (t2) is determined to be greater than the second analyte level threshold G2.

In some alternative aspects, the step 910 may instead include the processing circuitry 310 or 414 (e.g., the stream selector 506 of the processing circuitry 310 or 414), in determining whether to switch from use of the stream S2 of second analyte levels for display to use of the stream S1 of first analyte levels for display in the step 614 of the process 600, (1) determining whether the first analyte level for the second instance of the multiple instances of time is less than the second analyte level threshold G2 and then (2) determining whether the first analyte level for the third instance of the multiple instances of time is greater than the second analyte level threshold G2. In these alternative aspects, the DSS2 process 900A may include, subsequent to switching to use of the stream S2 of second analyte levels for display by proceeding to step 612, switching to use of the stream S1 of first analyte levels for display by proceeding from step 910 to step 608 if the first analyte level for the third instance of the multiple instances of time is determined to be greater than the second analyte level threshold G2. For example, as shown in FIG. 10D, subsequent to switching to use of the stream S2 of second analyte levels for display at the first instance of time (t1 of iteration 2), (1) the first analyte level for a second instance of time (a time between t1 and t2 of the second iteration) is determined to be less than the second analyte level threshold G2, which is 70 mg/dL in FIG. 10D, and then (2) the analyte monitoring system 100 switches to use of the stream S1 of first analyte levels for display at the third instance of time (t2 of iteration 2) because the first analyte level for the third instance of time (t2 of iteration 2) is determined to be greater than the second analyte level threshold G2.

In some aspects, as shown in FIG. 10B-10D, the second analyte level threshold G2 may be 70 mg/dL. In some alternative aspects, the second analyte level threshold G2 may be a different value (e.g., 65 mg/dL, 75 mg/dL, or 80 mg/dL).

FIG. 9B illustrates a third dynamic stream selection (DSS3) process 900B according to some aspects. In some aspects, the DSS3 process 900B may be performed in step 610 and/or step 614 of the process 600 shown in FIG. 6. In some aspects, one or more steps of the DSS3 process 900B may be performed by the processing circuitry 310 of the transceiver 104. In some alternative aspects, one or more steps of the DSS3 process 900B may be performed by the processing circuitry 414 of the display device 106. FIGS. 10A-10D illustrate examples of the DSS3 process 900B. As noted above, in FIGS. 10A-10D, the stream S1 is shown with a solid line when the stream S1 is not the selected stream of analyte values, and the stream S2 is shown with a long-dashed line when the stream S2 is not the selected stream of analyte value. In FIGS. 10A-10D, the selected stream of analyte values is shown as a bolded line. In the examples of FIGS. 10A, 10B, and 10D, selecting stream 2 at time t1 results in display of more accurate analyte levels calculated by the analyte monitoring system 100 (when compared to the venous blood analyte measurements) than if stream 1 were selected. In contrast, in the example of FIG. 10C, selecting stream 1 at time t1 results in display of more accurate analyte levels calculated by the analyte monitoring system 100 (when compared to the venous blood analyte measurements) than if stream 2 were selected.

In some aspects, as shown in FIG. 9B, the DSS3 process 900B may include, in determining whether to switch from use of the stream S1 of first analyte levels for display to use of the stream S2 of second analyte levels (including comparing the one or more of the first analyte levels to the one or more of the second analyte levels) in step 610 of the process 600, the steps 902, 904, and 906 and a step 907. In some aspects, the steps 902, 904, and 906 may be the same as in DSS2 process 900A of FIG. 9A. In some aspects, in the step 907, the processing circuitry 310 or 414 (e.g., the stream selector 506 of the processing circuitry 310 or 414) may determine whether a rate of change of the stream S1 of first analyte levels at the first instance of the multiple instances of time is less than or equal to a falling rate of change threshold. In some aspects, the falling rate of change threshold may be −0.3 mg/dL/min, which is equal to −18 mg/dL/hour. However, this is not required, and some alternative aspects may use a different falling rate of change threshold (e.g., −0.2 mg/dL/min or −0.4 mg/dL/min). In some aspects, the processing circuitry 310 or 414 (e.g., the stream selector 506 of the processing circuitry 310 or 414) may determine the rate of change of the stream S1 of first analyte levels using a least-squares fit. However, this is not required, and, in some alternative aspects, the rate of change may be determined in a different manner. For example, in some alternative aspects, the processing circuitry 310 or 414 (e.g., the stream selector 506 of the processing circuitry 310 or 414) may determine the rate of change of the stream S1 of first analyte levels using a weighted least squares fit, by calculating the rate of change that minimizes the L1 norm (e.g., using the least absolute deviation fit), or by calculating the slope from the first and last values in a series.

In some aspects, as shown in FIG. 9B, in the DSS3 process 900B, determining whether to switch from use of the stream S1 of first analyte levels for display to use of the stream S2 of second analyte levels for display in step 610 of the process 600 may include determining to not switch from the use of the stream S1 of first analyte levels for display to use of the stream S2 of second analyte levels for display (and returning to step 608 in which the stream selector 506 continues to select and the analyte monitoring system 100 continues to use the stream S1 of first analyte levels for display) if (a) the first analyte level for the first instance of time is not less than the first analyte level threshold G1 (step 902), (b) the first analyte level for the first instance of time is less than the second analyte level for the first instance of time (step 904), (c) the difference between the first and second analyte levels for the first instance of time is greater than the maximum analyte level difference threshold Δmax (step 906), and/or (d) the rate of change of the stream S1 of first analyte levels at the first instance of time is determined to be not less than or equal to the falling rate of change threshold (step 907). For example, as shown in FIG. 10C, the stream S1 of first analyte values remains the selected stream and continues to be used for display after the first instance of time (t1) because the first analyte level for the first instance of time is less than the second analyte level for the first instance of time (t1).

In some aspects, as shown in FIG. 9B, in the DSS3 process 900B, determining whether to switch from use of the stream S1 of first analyte levels for display to use of the stream S2 of second analyte levels for display in step 610 of the process 600 may include determining to switch from the use of the stream S1 of first analyte levels for display to use of the stream S2 of second analyte levels for display (and proceeding to step 612 in which the stream selector 506 selects and the analyte monitoring system 100 uses the stream S2 of second analyte levels for display) if (i) the first analyte level for the first instance of time is less than the first analyte level threshold G1, (ii) the first analyte level for the first instance of time is greater than the second analyte level for the first instance of time, (iii) the difference between the first and second analyte levels for the first instance of time is less than the analyte difference threshold Δmax, and (iv) the rate of change of the stream S1 of first analyte levels at the first instance of time is determined to be less than or equal to the falling rate of change threshold. For example, as shown in FIGS. 10A, 10B, and 10D, the stream S2 of second analyte levels is selected at the first instance of time (t1), and the analyte monitoring system 100 switches from using of the stream S1 of first analyte levels for display to use of the stream S2 of second analyte levels for display at the first instance of time (t1) because conditions (i) through (iv) are met.

In some aspects, as shown in FIG. 9B, the DSS3 process 900B may include, subsequent to using of the stream S2 of second analyte levels for display in step 612 of the process 600, a step 912 in which the processing circuitry 310 or 414 (e.g., the stream selector 506 of the processing circuitry 310 or 414), in determining whether to switch from use of the stream S2 of second analyte levels for display to use of the stream S1 of first analyte levels for display in the step 614 of the process 600, determines (1) whether the second analyte level for a second instance of the multiple instances of time is greater than the first analyte level threshold G1 and (2) whether a rate of change of the stream S2 of second analyte levels at the second instance of the multiple instances of time is greater than or equal to a rising rate of change threshold. In some aspects, the rising rate of change threshold may be +0.3 mg/dL/min, which is equal to +18 mg/dL/hour. However, this is not required, and some alternative aspects may use a different rising rate of change threshold (e.g., +0.2 mg/dL/min or +0.4 mg/dL/min). In some aspects, the processing circuitry 310 or 414 (e.g., the stream selector 506 of the processing circuitry 310 or 414) may determine the rate of change of the stream S2 of second analyte levels using a least-squares fit. However, this is not required, and, in some alternative aspects, the rate of change may be determined in a different manner. In some aspects, as shown in FIG. 9B, the DSS3 process 900B may include, subsequent to switching to use of the stream S2 of second analyte levels for display by proceeding to step 612, switching to use of the stream S1 of first analyte levels for display by proceeding from step 912 to step 608 if (i) the second analyte level for the second instance of the multiple instances of time is determined to be greater than the first analyte level threshold G1 and (ii) the rate of change of the stream S2 of second analyte levels at the second instance of the multiple instances of time is determined to be greater than or equal to the rising rate of change threshold.

In some alternative aspects, the step 912 may instead include the processing circuitry 310 or 414 (e.g., the stream selector 506 of the processing circuitry 310 or 414), in determining whether to switch from use of the stream S2 of second analyte levels for display to use of the stream S1 of first analyte levels for display in the step 614 of the process 600, (1) determining whether the first analyte level for the second instance of the multiple instances of time is greater than the first analyte level threshold G1 and (2) whether a rate of change of the stream S1 of first analyte levels at the second instance of the multiple instances of time is greater than or equal to the rising rate of change threshold. In these alternative aspects, the DSS3 process 900B may include, subsequent to switching to use of the stream S2 of second analyte levels for display by proceeding to step 612, switching to use of the stream S1 of first analyte levels for display by proceeding from step 908 to step 608 if (i) the first analyte level for the second instance of the multiple instances of time is determined to be greater than the first analyte level threshold G1 and (ii) the rate of change of the stream S1 of first analyte levels at the second instance of the multiple instances of time is determined to be greater than or equal to the rising rate of change threshold. For example, as shown in FIG. 10D, subsequent to switching to use of the stream S2 of second analyte levels for display at the first instance of time (t1 of iteration 1), the analyte monitoring system 100 switches to use of the stream S1 of first analyte levels for display at the second instance of time (t2 of iteration 1) because (i) the first analyte level for the second instance of time (t2 of iteration 1) is determined to be greater than the first analyte level threshold G1, which is 100 mg/dL in FIG. 10D, and (ii) the rate of change of the stream S1 of first analyte levels at the second instance time (t2 of iteration 1) is determined to be greater than or equal to the rising rate of change threshold.

In some aspects, as shown in FIG. 9B, the DSS3 process 900B may include, subsequent to using of the stream S2 of second analyte levels for display in step 612 of the process 600, a step 914 in which the processing circuitry 310 or 414 (e.g., the stream selector 506 of the processing circuitry 310 or 414), in determining whether to switch from use of the stream S2 of second analyte levels for display to use of the stream S1 of first analyte levels for display in the step 614 of the process 600, (1) determines whether the second analyte level for a second instance of the multiple instances of time is less than a second analyte level threshold G2 and then (2) if the second analyte level for the second instance of the multiple instances of time is determined to be less than the second analyte level threshold, (2A) determining whether the second analyte level for a third instance of the multiple instances of time is greater than the second analyte level threshold G2 and (2B) determining whether a rate of change of the stream S2 of second analyte levels at the third instance of the multiple instances of time is greater than or equal to a rising rate of change threshold. In some aspects, the rising rate of change threshold may be +0.3 mg/dL/min, which is equal to +18 mg/dL/hour. However, this is not required, and some alternative aspects may use a different rising rate of change threshold (e.g., +0.2 mg/dL/min or +0.4 mg/dL/min). In some aspects, the processing circuitry 310 or 414 (e.g., the stream selector 506 of the processing circuitry 310 or 414) may determine the rate of change of the stream S2 of second analyte levels using a least-squares fit. However, this is not required, and, in some alternative aspects, the rate of change may be determined in a different manner. In some aspects, as shown in FIG. 9B, the DSS3 process 900B may include, subsequent to switching to use of the stream S2 of second analyte levels for display by proceeding to step 612, switching to use of the stream S1 of first analyte levels for display by proceeding from step 914 to step 608 if (i) the second analyte level for the third instance of the multiple instances of time is determined to be greater than the second analyte level threshold G2 and (ii) the rate of change of the stream S2 of second analyte levels at the third instance of the multiple instances of time is determined to be greater than or equal to the rising rate of change threshold. For example, as shown in FIGS. 10A and 10B, subsequent to switching to use of the stream S2 of second analyte levels for display at the first instance of time (t1), (1) the second analyte level for a second instance of time (a time between t1 and t2) is determined to be less than the second analyte level threshold G2, which is 70 mg/dL in FIG. 10B, and then (2) the analyte monitoring system 100 switches to use of the stream S1 of first analyte levels for display at the third instance of time (t2) because (2A) the second analyte level for the third instance of time (t2) is determined to be greater than the second analyte level threshold G2 and (2B) the rate of change of the stream S2 of second analyte levels at the third instance of time (t2) is determined to be greater than or equal to the rising rate of change threshold.

In some alternative aspects, the step 914 may instead include the processing circuitry 310 or 414 (e.g., the stream selector 506 of the processing circuitry 310 or 414), in determining whether to switch from use of the stream S2 of second analyte levels for display to use of the stream S1 of first analyte levels for display in the step 614 of the process 600, (1) determining whether the first analyte level for the second instance of the multiple instances of time is less than the second analyte level threshold G2 and then (2A) determining whether the first analyte level for the third instance of the multiple instances of time is greater than the second analyte level threshold G2 and (2B) determining whether a rate of change of the stream S1 of first analyte levels at the third instance of the multiple instances of time is greater than or equal to a rising rate of change threshold. In these alternative aspects, the DSS2 process 900A may include, subsequent to switching to use of the stream S2 of second analyte levels for display by proceeding to step 612, switching to use of the stream S1 of first analyte levels for display by proceeding from step 914 to step 608 if (i) the first analyte level for the third instance of the multiple instances of time is determined to be greater than the second analyte level threshold G2 and (ii) the rate of change of the stream S1 of first analyte levels at the third instance of the multiple instances of time is determined to be greater than or equal to the rising rate of change threshold. For example, as shown in FIG. 10D, subsequent to switching to use of the stream S2 of second analyte levels for display at the first instance of time (t1 of iteration 2), (1) the first analyte level for a second instance of time (a time between t1 and t2 of the second iteration) is determined to be less than the second analyte level threshold G2, which is 70 mg/dL in FIG. 10D, and then (2) the analyte monitoring system 100 switches to use of the stream S1 of first analyte levels for display at the third instance of time (t2 of iteration 2) because (2A) the first analyte level for the third instance of time (t2 of iteration 2) is determined to be greater than the second analyte level threshold G2 and (2B) the rate of change of the stream S1 of first analyte levels at the third instance of time (t2 of iteration 2) is determined to be greater than or equal to the rising rate of change threshold.

FIG. 9C illustrates a fourth dynamic stream selection (DSS4) process 900C according to some aspects. In some aspects, the DSS4 process 900C may be performed in step 610 and/or step 614 of the process 600 shown in FIG. 6. In some aspects, one or more steps of the DSS4 process 900C may be performed by the processing circuitry 310 of the transceiver 104. In some alternative aspects, one or more steps of the DSS4 process 900C may be performed by the processing circuitry 414 of the display device 106. FIGS. 10A-10D illustrate examples of the DSS4 process 900C. As noted above, in FIGS. 10A-10D, the stream S1 is shown with a solid line when the stream S1 is not the selected stream of analyte values, and the stream S2 is shown with a long-dashed line when the stream S2 is not the selected stream of analyte value. In FIGS. 10A-10D, the selected stream of analyte values is shown as a bolded line. In the examples of FIGS. 10A, 10B, and 10D, selecting stream 2 at time t1 results in display of more accurate analyte levels calculated by the analyte monitoring system 100 (when compared to the venous blood analyte measurements) than if stream 1 were selected. In contrast, in the example of FIG. 10C, selecting stream 1 at time t₁ results in display of more accurate analyte levels calculated by the analyte monitoring system 100 (when compared to the venous blood analyte measurements) than if stream 2 were selected.

In some aspects, as shown in FIG. 9C, the DSS4 process 900C may include, in determining whether to switch from use of the stream S1 of first analyte levels for display to use of the stream S2 of second analyte levels (including comparing the one or more of the first analyte levels to the one or more of the second analyte levels) in step 610 of the process 600, steps 902, 904, 906b, and 907. In some aspects, the steps 902, 904, and 907 may be the same as in DSS3 process 900B of FIG. 9B. In some aspects, like in step 906 of the DSS2 and DSS3 processes 9A and 9B, in the step 906b, the processing circuitry 310 or 414 (e.g., the stream selector 506 of the processing circuitry 310 or 414) may determine whether a difference between the first and second analyte levels for the first instance of the multiple instances of time is greater than a maximum analyte level difference threshold Δmax. However, in some aspects, in step 906b, the processing circuitry 310 or 414 (e.g., the stream selector 506 of the processing circuitry 310 or 414) may additionally determine whether the difference between the first and second analyte levels for the first instance of the multiple instances of time is less than a minimum analyte level difference threshold Δmin. In some aspects, the minimum analyte level difference threshold Δmin may be 40 mg/dL. However, this is not required, and, in some alternative aspects, the minimum analyte level difference threshold Δmin may be a different value (e.g., 30 mg/dL, 35 mg/dL, 45 mg/dL, or 50 mg/dL).

In some aspects, as shown in FIG. 9C, in the DSS4 process 900C, determining whether to switch from use of the stream S1 of first analyte levels for display to use of the stream S2 of second analyte levels for display in step 610 of the process 600 may include determining to not switch from the use of the stream S1 of first analyte levels for display to use of the stream S2 of second analyte levels for display (and returning to step 608 in which the stream selector 506 continues to select and the analyte monitoring system 100 continues to use the stream S1 of first analyte levels for display) if (a) the first analyte level for the first instance of time is not less than the first analyte level threshold G1 (step 902), (b) the first analyte level for the first instance of time is less than the second analyte level for the first instance of time (step 904), (c) the difference between the first and second analyte levels for the first instance of time is greater than the maximum analyte level difference threshold Δmax (step 906b), (d) the difference between the first and second analyte levels for the first instance of time is less than the minimum analyte level difference threshold Δmin (step 906b), and/or (e) the rate of change of the stream S1 of first analyte levels at the first instance of time is determined to be not less than or equal to the falling rate of change threshold (step 907). For example, as shown in FIG. 10C, the stream S1 of first analyte values remains the selected stream and continues to be used for display after the first instance of time (t1) because the first analyte level for the first instance of time is less than the second analyte level for the first instance of time (t1).

In some aspects, as shown in FIG. 9C, in the DSS4 process 900C, determining whether to switch from use of the stream S1 of first analyte levels for display to use of the stream S2 of second analyte levels for display in step 610 of the process 600 may include determining to switch from the use of the stream S1 of first analyte levels for display to use of the stream S2 of second analyte levels for display (and proceeding to step 612 in which the stream selector 506 selects and the analyte monitoring system 100 uses the stream S2 of second analyte levels for display) if (i) the first analyte level for the first instance of time is less than the first analyte level threshold G1, (ii) the first analyte level for the first instance of time is greater than the second analyte level for the first instance of time, (iii) the difference between the first and second analyte levels for the first instance of time is less than the analyte difference threshold Δmax, (iv) the difference between the first and second analyte levels for the first instance of time is greater than the minimum analyte level difference threshold Δmin, and (v) the rate of change of the stream S1 of first analyte levels at the first instance of time is determined to be less than or equal to the falling rate of change threshold. For example, as shown in FIGS. 10A, 10B, and 10D, the stream S2 of second analyte levels is selected at the first instance of time (t1), and the analyte monitoring system 100 switches from using of the stream S1 of first analyte levels for display to use of the stream S2 of second analyte levels for display at the first instance of time (t1) because conditions (i) through (v) are met.

In some aspects, as shown in FIG. 9C, the DSS4 process 900C may include the steps 912 and 914 for determining whether to switch from use of the stream S2 of second analyte levels for display to use of the stream S1 of first analyte levels for display, which may be performed in the step 614 of the process 600.

FIG. 9D illustrates a fifth dynamic stream selection (DSS5) process 900D according to some aspects. In some aspects, the DSS5 process 900D may be performed in step 610 and/or step 614 of the process 600 shown in FIG. 6. In some aspects, one or more steps of the DSS5 process 900D may be performed by the processing circuitry 310 of the transceiver 104. In some alternative aspects, one or more steps of the DSS5 process 900D may be performed by the processing circuitry 414 of the display device 106. FIG. 10E illustrates an example of the DSS5 process 900D. In FIG. 10E, venous blood analyte measurements are labeled as YSI measurements, and the analyte levels calculated by the analyte monitoring system 100 for display are compared to the venous analyte measurements when assessing the accuracy of the analyte monitoring system 100. In the example of FIG. 10E, selecting stream 2 at each of the instances of time t1 results in display of more accurate analyte levels calculated by the analyte monitoring system 100 (when compared to the venous blood analyte measurements) than if stream 1 were selected. In contrast, in the example of FIG. 10C, selecting stream 1 at time t1 results in display of more accurate analyte levels calculated by the analyte monitoring system 100 (when compared to the venous blood analyte measurements) than if stream 2 were selected.

In some aspects, as shown in FIG. 9D, the DSS5 process 900D may include, in determining whether to switch from use of the stream S1 of first analyte levels for display to use of the stream S2 of second analyte levels (including comparing the one or more of the first analyte levels to the one or more of the second analyte levels) in step 610 of the process 600, steps 902, 904, 906b, 907, and 916. In some aspects, the steps 902, 904, 906b, and 907 may be the same as in DSS4 process 900C of FIG. 9C. In some aspects, as described above, the process 600 shown in FIG. 6 may include steps in which the analyte monitoring system 100 receives reference analyte values for respective reference instances of time. In some aspects, in the step 916, the processing circuitry 310 or 414 (e.g., the stream selector 506 of the processing circuitry 310 or 414) may determine whether a minimum cost of the stream S2 of second analyte levels at a last reference analyte value (minCost_S2) is too high. In some aspects, the step 916 may include comparing a minimum cost of the stream S1 of first analyte levels at the last reference analyte value (minCost_S1) to the minimum cost of the stream S2 of second analyte levels at the last reference analyte value (minCost_S2). In some aspects, the minimum cost (minCost) of a stream of analyte levels may be the value of the minimum of a cost function for the stream. In some aspects, the cost function may be evaluated at each reference instance of time over one or more parameters of the second method, which may be adjusted via calibration at each reference instance of time (e.g., using the received reference analyte values). In some aspects, the minimum cost may be related to the quality of optimization of the one or more parameters of the analyte level calculation method (e.g., the first method or the second method). In some aspects, the lower the minimum cost is the better (e.g., more successful) optimization was at the reference instance of time. In some aspects, evaluating whether a minimum cost of the stream S2 of second analyte levels at the last reference analyte value (minCost_S2) is too high in determining whether to switch from the stream S1 to the stream S2 may prevent a potential switch from stream S1 to stream S2 when stream S2 may have lower analyte values but may be less accurate than stream S1.

In some aspects, the step 916 may include comparing a minimum cost of the stream S1 of first analyte levels at the last reference analyte value (minCost_S1) to the minimum cost of the stream S2 of second analyte levels at the last reference analyte value (minCost_S2). In some aspects, the processing circuitry 310 or 414 (e.g., the stream selector 506 of the processing circuitry 310 or 414) may determine that the minimum cost of the stream S2 of second analyte levels at the last reference analyte value (minCost_S2) is too high if the minCost_S2 is not less than a sum of the minimum cost of the stream of first analyte levels at the last reference analyte value (minCost_S1) and a minimum cost difference threshold (ΔmC) (i.e., if minCost_S2>=minCost_S1+ΔmC). In some aspects, the minimum cost difference threshold (ΔmC) may be 0.25. However, this is not required, and, in some alternative aspects, the minimum cost difference threshold may be a different value (e.g., 0.20, 0.23, 0.27, or 0.30). In some aspects, the processing circuitry 310 or 414 (e.g., the stream selector 506 of the processing circuitry 310 or 414) may additionally or alternatively determine that the minimum cost of the stream S2 of second analyte levels at the last reference analyte value (minCost_S2) is too high if the minimum cost of the stream S2 of second analyte levels at the last reference analyte value (minCost_S2) is not less than a maximum cost threshold (mmc) (i.e., if minCost_S2>=mmc). In some aspects, the maximum cost threshold (mmc) may be 5.0. However, this is not required, and, in some alternative aspects, the minimum cost difference threshold may be a different value (e.g., 4.0, 4.5, 5.5, or 6.0).

In some aspects, as shown in FIG. 9D, in the DSS5 process 900D, determining whether to switch from use of the stream S1 of first analyte levels for display to use of the stream S2 of second analyte levels for display in step 610 of the process 600 may include determining to not switch from the use of the stream S1 of first analyte levels for display to use of the stream S2 of second analyte levels for display (and returning to step 608 in which the stream selector 506 continues to select and the analyte monitoring system 100 continues to use the stream S1 of first analyte levels for display) if (a) the first analyte level for the first instance of time is not less than the first analyte level threshold G1 (step 902), (b) the first analyte level for the first instance of time is less than the second analyte level for the first instance of time (step 904), (c) the difference between the first and second analyte levels for the first instance of time is greater than the maximum analyte level difference threshold Δmax (step 906b), (d) the difference between the first and second analyte levels for the first instance of time is less than the minimum analyte level difference threshold Δmin (step 906b), (e) the rate of change of the stream S1 of first analyte levels at the first instance of time is determined to be not less than or equal to the falling rate of change threshold (step 907), (f) the minimum cost of the stream S2 of second analyte levels at the last reference analyte value (minCost_S2) is not less than a sum of the minimum cost of the stream of first analyte levels at the last reference analyte value (minCost_S1) and the minimum cost difference threshold (ΔmC) (step 916), and/or (g) the minimum cost of the stream S2 of second analyte levels at the last reference analyte value (minCost_S2) is not less than the maximum cost threshold (mmc). For example, as shown in FIG. 10E, the stream S1 of first analyte values remains the selected stream and continues to be used for display after the first instance of time (t1) because of minCost values at the last calibration point.

In some aspects, as shown in FIG. 9D, in the DSS5 process 900D, determining whether to switch from use of the stream S1 of first analyte levels for display to use of the stream S2 of second analyte levels for display in step 610 of the process 600 may include determining to switch from the use of the stream S1 of first analyte levels for display to use of the stream S2 of second analyte levels for display (and proceeding to step 612 in which the stream selector 506 selects and the analyte monitoring system 100 uses the stream S2 of second analyte levels for display) if (i) the first analyte level for the first instance of time is less than the first analyte level threshold G1, (ii) the first analyte level for the first instance of time is greater than the second analyte level for the first instance of time, (iii) the difference between the first and second analyte levels for the first instance of time is less than the analyte difference threshold Δmax, (iv) the difference between the first and second analyte levels for the first instance of time is greater than the minimum analyte level difference threshold Δmin, (v) the rate of change of the stream S1 of first analyte levels at the first instance of time is determined to be less than or equal to the falling rate of change threshold, (vi) the minimum cost of the stream S2 of second analyte levels at the last reference analyte value (minCost_S2) is less than a sum of the minimum cost of the stream of first analyte levels at the last reference analyte value (minCost_S1) and the minimum cost difference threshold (ΔmC) (step 916), and (vii) the minimum cost of the stream S2 of second analyte levels at the last reference analyte value (minCost_S2) is not less than the maximum cost threshold (mmc).

In some aspects, as shown in FIG. 9D, the DSS5 process 900D may include the steps 912 and 914 for determining whether to switch from use of the stream S2 of second analyte levels for display to use of the stream S1 of first analyte levels for display, which may be performed in the step 614 of the process 600.

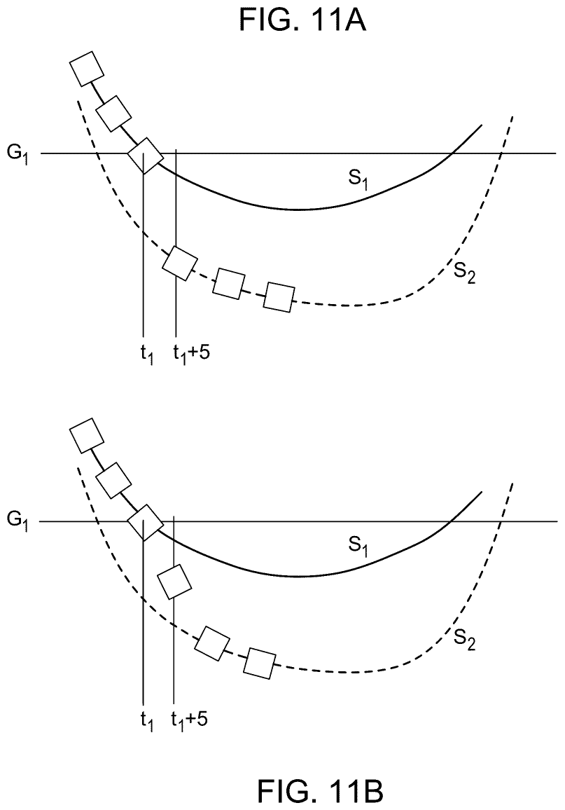

In some aspects, as shown in FIG. 11A, switching to use of the stream S2 of second analyte levels for display (e.g., by proceeding from step 610 to step 612 in FIG. 6, from step 706 to step 612 in FIG. 7, from step 906 to step 612 in FIG. 9A, from step 907 to step 612 in FIG. 9B or 9C, or from step 916 to step 612 in FIG. 9D) may include an immediate switch to the stream S2 of second analyte levels for display such that the analyte monitoring system 100 uses the stream S1 of first analyte levels at the one instance (t1) of the of the multiple instances of time and then uses the stream S2 of second analyte levels for display at the next instance (t1+5) of the of the multiple instances of time. In some alternative aspects, as shown in FIG. 11B, switching to use of the stream S2 of second analyte levels for display may include using an average of the first and second analyte levels for one or more transitionary instances (e.g., t1+5 minutes) of the multiple instances of time for display and then using the second analyte levels for one or more instances of the multiple instances of time subsequent to the one or more transitionary instances (e.g., t1+10 minutes and t1+15 minutes).

In some aspects, switching to use of the stream S1 of first analyte levels for display (e.g., by proceeding from step 614 to step 608 in FIG. 6, from step 706 to step 608 in FIG. 7, from step 908 or 910 to step 608 in FIG. 9A, or from step 912 or 914 to step 608 in any of FIGS. 9B-9D) may include an immediate switch to the stream S1 of first analyte levels for display such that the analyte monitoring system 100 uses the stream S2 of second analyte levels at the one instance of the of the multiple instances of time and then uses the stream S1 of first analyte levels for display at the next instance of the of the multiple instances of time. In some alternative aspects, switching to use of the stream S1 of first analyte levels for display may include using an average of the first and second analyte levels for one or more transitionary instances of the multiple instances of time for display and then using the first analyte levels for one or more instances of the multiple instances of time subsequent to the one or more transitionary instances.

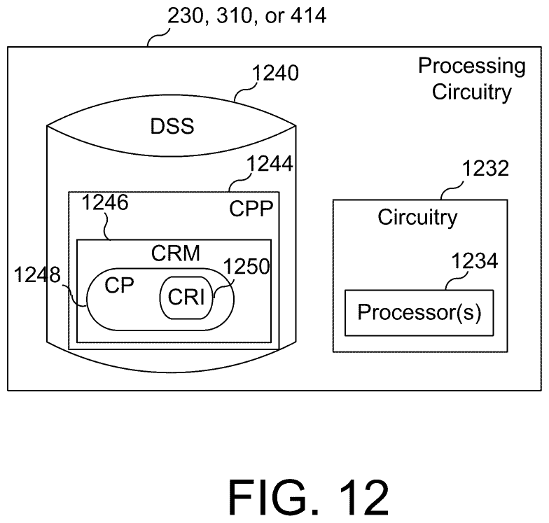

FIG. 12 is a block diagram of an aspect of processing circuitry (e.g., the processing circuitry 230 of the analyte sensor 102, the processing circuitry 310 of the transceiver 104, and/or the processing circuitry 414 of the display device 106) of the analyte monitoring system 100. As shown in FIG. 12, in some aspects, the processing circuitry may include circuitry 1232 (e.g., one or more circuits), such as an application specific integrated circuit (ASIC), field-programmable gate arrays (FPGAs), a logic circuit, and the like. In some aspects, the circuitry 1232 may include one or more processors 1234 (e.g., one or more general purpose microprocessors). In some aspects, the processing circuitry may include a data storage system (DSS) 1240. The DSS 1240 may include one or more non-volatile storage devices and/or one or more volatile storage devices (e.g., random access memory (RAM)). In aspects where the processing circuitry includes circuitry 1232, the DSS 1240 may include a computer program product (CPP) 1244. CPP 1244 may include or be a computer readable medium (CRM) 1246. The CRM 1246 may store a computer program (CP) 1248 comprising computer readable instructions (CRI) 1250. In some aspects in which the processing circuitry is the processing circuitry 414 of the display device 106, the CRM 1246 may store, among other programs, the MMA, and the CRI 1250 may include one or more instructions of the MMA. The CRM 1246 may be a non-transitory computer readable medium, such as, but not limited, to magnetic media (e.g., a hard disk), optical media (e.g., a DVD), solid state devices (e.g., random access memory (RAM) or flash memory), and the like. In some aspects, the CRI 1250 of computer program 1248 may be configured such that when executed by circuitry 1232, the CRI 1250 causes the computer to perform steps described above (e.g., steps described above with reference to processes 600, 700, 900A, 900B, 900C, and 900D). In other aspects, the processing circuitry may be configured to perform steps described herein without the need for a computer program. That is, for example, the processing circuitry may consist merely of one or more ASICs. Hence, the features of the aspects described herein may be implemented in hardware and/or software.

Aspects of the present invention have been fully described above with reference to the drawing figures. Although the invention has been described based upon these preferred aspects, it would be apparent to those of skill in the art that certain modifications, variations, and alternative constructions could be made to the described aspects within the spirit and scope of the invention. For example, although the aspects of the invention in which the analyte indicator molecules 206 and interferent indicator molecules 208 are distributed throughout one or more analyte and/or interferent indicators 204, this is not required. In some alternative aspects, the analyte and/or interferent indicators 204 of a sensing device of the analyte sensor 100 may include an analyte indicator including analyte indicator molecules 206 and a separate and distinct interferent indicator including interferent indicator molecules 208. In these alternative aspects, the analyte indicator 207 and the interferent indicator 209 may be spatially separated from one another.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel. For example, steps 604 and 606 of the process 600 of FIG. 6 could alternatively be performed in a different order or in parallel, steps 702 and 704 of the process 700 of FIG. 7 could alternatively be performed in a different order or in parallel, steps 902, 904, and 906 of the process 900A of FIG. 9A could alternatively be performed in a different order or in parallel, steps 902, 904, 906, and 907 of the process 900B of FIG. 9B could alternatively be performed in a different order or in parallel, steps 902, 904, 906b, and 907 of the process 900C of FIG. 9C could alternatively be performed in a different order or in parallel, and steps 902, 904, 906b, 907, and 916 of the process 900D of FIG. 9D could alternatively be performed in a different order or in parallel.

### Summary of Embodiments

A1. A method comprising: receiving sensor data for multiple instances of time, wherein the sensor data was conveyed by an analyte sensor; using a first method to calculate a stream of first analyte levels for the multiple instances of time based on at least the sensor data for the multiple instances of time; using a second method to calculate a stream of second analyte levels for the multiple instances of time based on at least the sensor data for the multiple instances of time; using the stream of first analyte levels for display; determining whether to switch from use of the stream of first analyte levels for display to use of the stream of second analyte levels for display, wherein determining whether to switch from use of the stream of first analyte levels for display to use of the stream of second analyte levels comprises comparing one or more of the first analyte levels to one or more of the second analyte levels; continuing to use the stream of first analyte levels for display if determined to not switch from use of the stream of first analyte levels to use of the stream of second analyte levels; and switching to use of the stream of second analyte levels for display if determined to switch from use of the stream of first analyte levels to use of the stream of second analyte levels.

A2. The method of embodiment A1, wherein the first method is one of a ratio method and a two-parameter method, and the second method is another of the ratio method and the two-parameter method.

A3. The method of embodiment A1 or A2, wherein using the stream of first analyte levels for display comprises displaying one or more of the first analyte levels.

A4. The method of any one of embodiments A1-A3, wherein using the stream of first analyte levels for display comprises conveying one or more of the first analyte levels to a display device for display by the display device.

A5. The method of any one of embodiment A1-A4, further comprising: receiving a first reference analyte value for a first reference instance of time; and receiving a second reference analyte value for a second reference instance of time; wherein determining whether to switch from use of the stream of first analyte levels for display to use of the stream of second analyte levels for display is performed if the second reference analyte value is received.

A6. The method of embodiment A5, wherein comparing the one or more of the first analyte levels to the one or more of the second analyte levels includes: determining an aggregate value of the first analyte levels that are for instances of the multiple instances of time that are between the first and second reference instances of time; determining an aggregate value of the second analyte levels that are for instances of the multiple instances of time that are between the first and second reference instances of time; and determining whether the aggregate value of the second analyte levels is lower than the aggregate value of the first analyte levels.

A7. The method of embodiment A6, wherein determining whether to switch from use of the stream of first analyte levels for display to use of the stream of second analyte levels for display comprises determining to not switch from the use of the stream of first analyte levels for display to use of the stream of second analyte levels for display if the aggregate value of the second analyte levels is not determined to be lower than the aggregate value of the first analyte levels; and determining whether to switch from use of the stream of first analyte levels for display to use of the stream of second analyte levels for display comprises determining to switch from the use of the stream of first analyte levels for display to use of the stream of second analyte levels for display if the aggregate value of the second analyte levels is determined to be lower than the aggregate value of the first analyte levels.

A8. The method of embodiment A6 or A7, wherein the determined aggregate value of the first analyte levels that are for the instances of the multiple instances of time that are between the first and second reference instances of time is a first quartile of the first analyte levels that are for the instances of the multiple instances of time that are between the first and second reference instances of time, and the determined aggregate value of the second analyte levels that are for the instances of the multiple instances of time that are between the first and second reference instances of time is a first quartile of the second analyte levels that are for the instances of the multiple instances of time that are between the first and second reference instances of time.

A9. The method of any one of embodiments A5-A8, further comprising: receiving a third reference analyte value for a third reference instance of time; determining a second aggregate value of the first analyte levels that are for instances of the multiple instances of time that are between the second and third reference instances of time; determining a second aggregate value of the second analyte levels that are for instances of the multiple instances of time that are between the second and third reference instances of time; determining whether the second aggregate value of the second analyte levels is lower than the second aggregate value of the first analyte levels; using the stream of first analyte levels for display for one or more instances of the multiple instances of time that are subsequent to the third reference instances of time if the second aggregate value of the second analyte levels is not determined to be lower than the second aggregate value of the first analyte levels; and using the stream of second analyte levels for display for the one or more instances of the multiple instances of time that are subsequent to the third reference instances of time if the second aggregate value of the second analyte levels is determined to be lower than the second aggregate value of the first analyte levels.

A10. The method of any one of embodiments A1-A4, wherein determining whether to switch from use of the stream of first analyte levels for display to use of the stream of second analyte levels comprises (a) determining whether the first analyte level for a first instance of the multiple instances of time is less than a first analyte level threshold and/or (b) determining at the first instance of the multiple instances of time whether a predicted first analyte level is less than the first analyte level threshold; wherein comparing the one or more of the first analyte levels to the one or more of the second analyte levels includes determining whether the second analyte level for the first instance of the multiple instances of time is lower than the first analyte level for the first instance of the multiple instances of time.

A11. The method of embodiment A10, wherein comparing the one or more of the first analyte levels to the one or more of the second analyte levels further includes determining whether a difference between the first and second analyte levels for the first instance of the multiple instances of time is greater than a maximum analyte level difference threshold.

A12. The method of embodiment A10 or A11, wherein determining whether to switch from use of the stream of first analyte levels for display to use of the stream of second analyte levels comprises determining whether the first analyte level for the first instance of the multiple instances of time is less than the first analyte level threshold.

A13. The method of any one of embodiments A10-A12, wherein determining whether to switch from use of the stream of first analyte levels for display to use of the of the stream of second analyte levels for display comprises determining at the first instance of the multiple instances of time whether the predicted first analyte level is less than the first analyte level threshold.

A14. The method of any of embodiments A11-A13, wherein determining whether to switch from use of the stream of first analyte levels for display to use of the stream of second analyte levels for display comprises determining to not switch from the use of the stream of first analyte levels for display to use of the stream of second analyte levels for display if (a) the first analyte level for the first instance of time of the multiple instances of time is less than the second analyte level for the first instance of time and/or (b) the difference between the first and second analyte levels for the first instance of time is greater than the maximum analyte level difference threshold.

A15. The method of any one of embodiments A10-A14, wherein determining whether to switch from use of the stream of first analyte levels for display to use of the stream of second analyte levels for display comprises determining to switch from the use of the stream of first analyte levels for display to use of the stream of second analyte levels for display if (i) the first analyte level for the first instance of time is greater than the second analyte level for the first instance of time and (ii) the difference between the first and second analyte levels for the first instance of time is less than the analyte difference threshold.

A16. The method of any one of embodiments A10-A15, wherein determining whether to switch from use of the stream of first analyte levels for display to use of the stream of second analyte levels for display further comprises determining whether a rate of change of the stream of first analyte levels at the first instance of the multiple instances of time is less than or equal to a falling rate of change threshold.

A17. The method of embodiment A16, wherein determining whether to switch from use of the stream of first analyte levels for display to use of the stream of second analyte levels for display comprises determining to not switch from the use of the stream of first analyte levels for display to use of the stream of second analyte levels for display if the rate of change of the stream of first analyte levels at the first instance of the multiple instances of time is determined to be not less than or equal to the falling rate of change threshold.

A18. The method of any one of embodiments A10-A17, wherein comparing the one or more of the first analyte levels to the one or more of the second analyte levels further includes determining whether the difference between the first and second analyte levels for the first instance of the multiple instances of time is less than a minimum analyte level difference threshold.

A19. The method of embodiment A18, wherein determining whether to switch from use of the stream of first analyte levels for display to use of the stream of second analyte levels for display comprises determining to not switch from the use of the stream of first analyte levels for display to use of the stream of second analyte levels for display if the difference between the first and second analyte levels for the first instance of the multiple instances of time is determined to be less than the minimum analyte level difference threshold.

A20. The method of embodiment A18 or A19, wherein the minimum analyte level difference threshold is 40 mg/dL.

A21. The method of any one of embodiments A10-A20, wherein comparing the one or more of the first analyte levels to the one or more of the second analyte levels further includes comparing a minimum cost of the stream of first analyte levels at a last reference analyte value to a minimum cost of the stream of second analyte levels at the last reference analyte value.

A22. The method of embodiment A21, wherein determining whether to switch from use of the stream of first analyte levels for display to use of the stream of second analyte levels for display comprises determining to not switch from the use of the stream of first analyte levels for display to use of the stream of second analyte levels for display if the minimum cost of the stream of second analyte levels at the last reference analyte value is not less than a sum of the minimum cost of the stream of first analyte levels at the last reference analyte value and a minimum cost difference threshold.

A23. The method of any one of embodiments A10-A22, wherein determining whether to switch from use of the stream of first analyte levels for display to use of the stream of second analyte levels for display further comprises determining whether a minimum cost of the stream of second analyte levels at the last reference analyte value is less than a maximum cost threshold.

A24. The method of embodiment A23, wherein determining whether to switch from use of the stream of first analyte levels for display to use of the stream of second analyte levels for display comprises determining to not switch from the use of the stream of first analyte levels for display to use of the stream of second analyte levels for display if the minimum cost of the stream of second analyte levels at the last reference analyte value is determined to be not less than the maximum cost threshold.

A25. The method of any one of embodiments A10-A24, further comprising, subsequent to switching to use of the stream of second analyte levels for display: determining whether the first analyte level for a second instance of the multiple instances of time is greater than the first analyte level threshold; and switching to use of the stream of first analyte levels for display if the first analyte level for the second instance of the multiple instances of time is determined to be greater than the first analyte level threshold.

A26. The method of any one of embodiments A10-A24, further comprising, subsequent to switching to use of the stream of second analyte levels for display: determining whether the second analyte level for a second instance of the multiple instances of time is greater than the first analyte level threshold; and switching to use of the stream of first analyte levels for display if the second analyte level for the second instance of the multiple instances of time is determined to be greater than the first analyte level threshold.

A27. The method of any one of embodiments A10-A24, further comprising, subsequent to switching to use of the stream of second analyte levels for display: determining whether the first analyte level for a second instance of the multiple instances of time is greater than the first analyte level threshold; determining whether a rate of change of the stream of first analyte levels at the second instance of the multiple instances of time is greater than or equal to a rising rate of change threshold; and switching to use of the stream of first analyte levels for display if (i) the first analyte level for the second instance of the multiple instances of time is determined to be greater than the first analyte level threshold and (ii) the rate of change of the stream of first analyte levels at the second instance of the multiple instances of time is determined to be greater than or equal to the rising rate of change threshold.

A28. The method of any one of embodiments A10-A24, further comprising, subsequent to switching to use of the stream of second analyte levels for display: determining whether the second analyte level for a second instance of the multiple instances of time is greater than the first analyte level threshold; determining whether a rate of change of the stream of second analyte levels at the second instance of the multiple instances of time is greater than or equal to a rising rate of change threshold; and switching to use of the stream of first analyte levels for display if (i) the second analyte level for the second instance of the multiple instances of time is determined to be greater than the first analyte level threshold and (ii) the rate of change of the stream of second analyte levels at the second instance of the multiple instances of time is determined to be greater than or equal to the rising rate of change threshold.

A29. The method of any one of embodiments A10-A28, further comprising, subsequent to switching to use of the stream of second analyte levels for display: determining whether the first analyte level for a second instance of the multiple instances of time is less than a second analyte level threshold; if the first analyte level for the second instance of the multiple instances of time is determined to be less than the second analyte level threshold, determining whether the first analyte level for a third instance of the multiple instances of time is greater than the second analyte level threshold; and switching to use of the stream of first analyte levels for display if the first analyte level for the third instance of the multiple instances of time is determined to be greater than the second analyte level threshold.

A30. The method of any one of embodiments A10-A28, further comprising, subsequent to switching to use of the stream of second analyte levels for display: determining whether the second analyte level for a second instance of the multiple instances of time is less than a second analyte level threshold; if the second analyte level for the second instance of the multiple instances of time is determined to be less than the second analyte level threshold, determining whether the second analyte level for a third instance of the multiple instances of time is greater than the second analyte level threshold; and switching to use of the stream of first analyte levels for display if the second analyte level for the third instance of the multiple instances of time is determined to be greater than the second analyte level threshold.

A31 The method of any one of embodiments A10-A28, further comprising, subsequent to switching to use of the stream of second analyte levels for display: determining whether the first analyte level for a second instance of the multiple instances of time is less than a second analyte level threshold; if the first analyte level for the second instance of the multiple instances of time is determined to be less than the second analyte level threshold, (i) determining whether the first analyte level for a third instance of the multiple instances of time is greater than the second analyte level threshold and (ii) determining whether a rate of change of the stream of first analyte levels at the third instance of the multiple instances of time is greater than or equal to a rising rate of change threshold; and switching to use of the stream of first analyte levels for display if (i) the first analyte level for the third instance of the multiple instances of time is determined to be greater than the second analyte level threshold and (ii) the rate of change of the stream of first analyte levels at the third instance of the multiple instances of time is determined to be greater than or equal to the rising rate of change threshold.

A32. The method of any one of embodiments A10-A26, further comprising, subsequent to switching to use of the stream of second analyte levels for display: determining whether the second analyte level for a second instance of the multiple instances of time is less than a second analyte level threshold; if the second analyte level for the second instance of the multiple instances of time is determined to be less than the second analyte level threshold, (i) determining whether the second analyte level for a third instance of the multiple instances of time is greater than the second analyte level threshold and (ii) determining whether a rate of change of the stream of second analyte levels at the third instance of the multiple instances of time is greater than or equal to a rising rate of change threshold; and switching to use of the stream of first analyte levels for display if (i) the second analyte level for the third instance of the multiple instances of time is determined to be greater than the second analyte level threshold and (ii) the rate of change of the stream of second analyte levels at the third instance of the multiple instances of time is determined to be greater than or equal to the rising rate of change threshold.

A33. The method of any one of embodiments A29-32, wherein the second analyte level threshold is 70 mg/dL.

A34. The method of any one of embodiments A29-A32, wherein the second analyte level threshold is 75 mg/dL.

A35. The method of any one of embodiments A10-A34, wherein the first analyte level threshold is 100 mg/dL.

A36. The method of any one of embodiments A10-A34, wherein the first analyte level threshold is 110 mg/dL.

A37. The method of any one of embodiments A10-A36, wherein the maximum analyte level difference threshold is 40 mg/dL.

A38. The method of any of embodiments A1-A37, wherein switching to use of the stream of second analyte levels for display comprises using an average of the first and second analyte levels for one or more transitionary instances of the multiple instances of time for display and then using the second analyte levels for one or more instances of the multiple instances of time subsequent to the one or more transitionary instances.

A39. The method of any one of embodiments A25-A38, wherein switching to use of the stream of first analyte levels for display comprises using an average of the first and second analyte levels for one or more transitionary instances of the multiple instances of time for display and then using the first analyte levels for one or more instances of the multiple instances of time subsequent to the one or more transitionary instances.

B40. An apparatus comprising: an antenna configured to receive sensor data for multiple instances of time, wherein the sensor data is conveyed by an analyte sensor; and processing circuitry configured to: use a first method to calculate a stream of first analyte levels for the multiple instances of time based on at least the sensor data for the multiple instances of time; use a second method to calculate a stream of second analyte levels for the multiple instances of time based on at least the sensor data for the multiple instances of time; use the stream of first analyte levels for display; determine whether to switch from use of the stream of first analyte levels for display to use of the stream of second analyte levels for display, wherein determining whether to switch from use of the stream of first analyte levels for display to use of the stream of second analyte levels comprises comparing one or more of the first analyte levels to one or more of the second analyte levels; continue to use the stream of first analyte levels for display if determined to not switch from use of the stream of first analyte levels to use of the stream of second analyte levels; and switch to use of the stream of second analyte levels for display if determined to switch from use of the stream of first analyte levels to use of the stream of second analyte levels.

B41. The apparatus of embodiment B40, wherein the first method is one of a ratio method and a two-parameter method, and the second method is another of the ratio method and the two-parameter method.

B42. The apparatus of embodiment B40 or B41, wherein the apparatus further comprises a display, and the processing circuitry is further configured to, in using the stream of first analyte levels for display, cause the display to display the one or more of the first analyte levels.

B43. The apparatus of any one of embodiments B40-B42, wherein the antenna is a first antenna, the apparatus further comprises a second antenna, and the processing circuitry is further configured to, in using the stream of first analyte levels for display, cause the second antenna to convey one or more of the first analyte levels to a display device for display by the display device.

B44. The apparatus of any one of embodiments B40-B43, wherein the processing circuitry is further configured to: receive a first reference analyte value for a first reference instance of time; and receive a second reference analyte value for a second reference instance of time; wherein the processing circuitry is configured to perform determining whether to switch from use of the stream of first analyte levels for display to use of the stream of second analyte levels for display if the second reference analyte value is received.

B45. The apparatus of embodiment B44, wherein, in comparing the one or more of the first analyte levels to the one or more of the second analyte levels, the processing circuitry is further configured to: determine an aggregate value of the first analyte levels that are for instances of the multiple instances of time that are between the first and second reference instances of time; determine an aggregate value of the second analyte levels that are for instances of the multiple instances of time that are between the first and second reference instances of time; and determine whether the aggregate value of the second analyte levels is lower than the aggregate value of the first analyte levels.

B46. The apparatus of embodiment B45, wherein the processing circuitry is further configured to, in determining whether to switch from use of the stream of first analyte levels for display to use of the stream of second analyte levels for display, determine to not switch from the use of the stream of first analyte levels for display to use of the stream of second analyte levels for display if the aggregate value of the second analyte levels is not determined to be lower than the aggregate value of the first analyte levels; and the processing circuitry is further configured to, in determining whether to switch from use of the stream of first analyte levels for display to use of the stream of second analyte levels for display, determine to switch from the use of the stream of first analyte levels for display to use of the stream of second analyte levels for display if the aggregate value of the second analyte levels is determined to be lower than the aggregate value of the first analyte levels.

B47. The apparatus of embodiment B45 or B46, wherein the determined aggregate value of the first analyte levels that are for the instances of the multiple instances of time that are between the first and second reference instances of time is a first quartile of the first analyte levels that are for the instances of the multiple instances of time that are between the first and second reference instances of time, and the determined aggregate value of the second analyte levels that are for the instances of the multiple instances of time that are between the first and second reference instances of time is a first quartile of the second analyte levels that are for the instances of the multiple instances of time that are between the first and second reference instances of time.

B48. The apparatus of any one of embodiments B44-B47, wherein the apparatus is further configured to receive a third reference analyte value for a third reference instance of time; and the processing circuitry is further configured to: determine a second aggregate value of the first analyte levels that are for instances of the multiple instances of time that are between the second and third reference instances of time; determine a second aggregate value of the second analyte levels that are for instances of the multiple instances of time that are between the second and third reference instances of time; determine whether the second aggregate value of the second analyte levels is lower than the second aggregate value of the first analyte levels; use the stream of first analyte levels for display for one or more instances of the multiple instances of time that are subsequent to the third reference instances of time if the second aggregate value of the second analyte levels is not determined to be lower than the second aggregate value of the first analyte levels; and use the stream of second analyte levels for display for the one or more instances of the multiple instances of time that are subsequent to the third reference instances of time if the second aggregate value of the second analyte levels is determined to be lower than the second aggregate value of the first analyte levels.

B49. The apparatus of any one of embodiment B40-B43, wherein, in determining whether to switch from use of the stream of first analyte levels for display to use of the stream of second analyte levels, the processing circuitry is configured to: (a) determine whether the first analyte level for a first instance of the multiple instances of time is less than a first analyte level threshold and/or (b) determine at the first instance of the multiple instances of time whether a predicted first analyte level is less than the first analyte level threshold; wherein, in comparing the one or more of the first analyte levels to the one or more of the second analyte levels, the processing circuitry is further configured to determine whether the second analyte level for a first instance of the multiple instances of time is lower than the first analyte level for the first instance of the multiple instances of time.

B50. The apparatus of embodiment B49, wherein, in comparing the one or more of the first analyte levels to the one or more of the second analyte levels, the processing circuitry is further configured to determine whether a difference between the first and second analyte levels for the first instance of the multiple instances of time is greater than a maximum analyte level difference threshold.

B51. The apparatus of embodiment B49 or B50, wherein the processing circuitry is further configured to, in determining whether to switch from use of the stream of first analyte levels for display to use of the stream of second analyte levels, determine whether the first analyte level for the first instance of the multiple instances of time is less than the first analyte level threshold.

B52. The apparatus of any one of embodiments B49-B51, wherein the processing circuitry is configured to, in determining whether to switch from use of the stream of first analyte levels for display to use of the of the stream of second analyte levels for display, determine at the first instance of the multiple instances of time whether the predicted first analyte level is less than the first analyte level threshold.

B53. The apparatus of any of embodiments B50-B52, wherein the processing circuitry is further configured to, in determining whether to switch from use of the stream of first analyte levels for display to use of the stream of second analyte levels for display, determine to not switch from the use of the stream of first analyte levels for display to use of the stream of second analyte levels for display if (a) the first analyte level for the first instance of the multiple instances of time is less than the second analyte level for the first instance of time and/or (b) the difference between the first and second analyte levels for the first instance of time is greater than the maximum analyte level difference threshold.

B54. The apparatus of any one of embodiments B49-B53, wherein the processing circuitry is configured to, in determining whether to switch from use of the stream of first analyte levels for display to use of the stream of second analyte levels for display, determine to switch from the use of the stream of first analyte levels for display to use of the stream of second analyte levels for display if (i) the first analyte level for the first instance of time is greater than the second analyte level for the second instance of time and (ii) the difference between the first and second analyte levels for the first instance of time is less than the analyte difference threshold.

B55. The apparatus of any one of embodiments B49-B54, wherein the processing circuitry is configured to, in determining whether to switch from use of the stream of first analyte levels for display to use of the stream of second analyte levels for display, determine whether a rate of change of the stream of first analyte levels at the first instance of the multiple instances of time is less than or equal to a falling rate of change threshold.

B56. The apparatus of embodiment B55, wherein the processing circuitry is configured to, in determining whether to switch from use of the stream of first analyte levels for display to use of the stream of second analyte levels for display, determine to not switch from the use of the stream of first analyte levels for display to use of the stream of second analyte levels for display if the rate of change of the stream of first analyte levels at the first instance of the multiple instances of time is determined to be not less than or equal to the falling rate of change threshold.

B57. The apparatus of any one of embodiments B49-B56, wherein the processing circuitry is configured to, in comparing the one or more of the first analyte levels to the one or more of the second analyte levels, determine whether the difference between the first and second analyte levels for the first instance of the multiple instances of time is less than a minimum analyte level difference threshold.

B58. The apparatus of embodiment B57, wherein the processing circuitry is configured to, in determining whether to switch from use of the stream of first analyte levels for display to use of the stream of second analyte levels for display, determine to not switch from the use of the stream of first analyte levels for display to use of the stream of second analyte levels for display if the difference between the first and second analyte levels for the first instance of the multiple instances of time is determined to be less than the minimum analyte level difference threshold.

B59. The apparatus of embodiment B57 or B58, wherein the minimum analyte level difference threshold is 40 mg/dL.

B60. The apparatus of any one of embodiments B49-B59, wherein the processing circuitry is configured to, in comparing the one or more of the first analyte levels to the one or more of the second analyte levels, compare a minimum cost of the stream of first analyte levels at a last reference analyte value to a minimum cost of the stream of second analyte levels at the last reference analyte value.

B61. The apparatus of embodiment B60, wherein the processing circuitry is configured to, in determining whether to switch from use of the stream of first analyte levels for display to use of the stream of second analyte levels for display, determine to not switch from the use of the stream of first analyte levels for display to use of the stream of second analyte levels for display if the minimum cost of the stream of second analyte levels at the last reference analyte value is not less than a sum of the minimum cost of the stream of first analyte levels at the last reference analyte value and a minimum cost difference threshold.

B62. The apparatus of any one of embodiments B49-B61, wherein the processing circuitry is configured to, in determining whether to switch from use of the stream of first analyte levels for display to use of the stream of second analyte levels for display, determine whether a minimum cost of the stream of second analyte levels at the last reference analyte value is less than a maximum cost threshold.

B63. The apparatus of embodiment B62, wherein the processing circuitry is configured to, in determining whether to switch from use of the stream of first analyte levels for display to use of the stream of second analyte levels for display, determine to not switch from the use of the stream of first analyte levels for display to use of the stream of second analyte levels for display if the minimum cost of the stream of second analyte levels at the last reference analyte value is determined to be not less than the maximum cost threshold.

B64. The apparatus of any one of embodiments B49-B63, wherein the processing circuitry is further configured to, subsequent to switching to use of the stream of second analyte levels for display: determine whether the first analyte level for a second instance of the multiple instances of time is greater than the first analyte level threshold; and switch to use of the stream of first analyte levels for display if the first analyte level for the second instance of the multiple instances of time is determined to be greater than the first analyte level threshold.

B65. The apparatus of any one of embodiments B49-B63, wherein the processing circuitry is further configured to, subsequent to switching to use of the stream of second analyte levels for display: determine whether the second analyte level for a second instance of the multiple instances of time is greater than the first analyte level threshold; and switch to use of the stream of first analyte levels for display if the second analyte level for the second instance of the multiple instances of time is determined to be greater than the first analyte level threshold.

B66. The apparatus of any one of embodiments B49-B63, wherein the processing circuitry is further configured to, subsequent to switching to use of the stream of second analyte levels for display: determine whether the first analyte level for a second instance of the multiple instances of time is greater than the first analyte level threshold; determine whether a rate of change of the stream of first analyte levels at the second instance of the multiple instances of time is greater than or equal to a rising rate of change threshold; and switch to use of the stream of first analyte levels for display if (i) the first analyte level for the second instance of the multiple instances of time is determined to be greater than the first analyte level threshold and (ii) the rate of change of the stream of first analyte levels at the second instance of the multiple instances of time is determined to be greater than or equal to the rising rate of change threshold.

B67. The apparatus of any one of embodiments B49-B63, wherein the processing circuitry is further configured to, subsequent to switching to use of the stream of second analyte levels for display: determine whether the second analyte level for a second instance of the multiple instances of time is greater than the first analyte level threshold; determine whether a rate of change of the stream of second analyte levels at the second instance of the multiple instances of time is greater than or equal to a rising rate of change threshold; and switch to use of the stream of first analyte levels for display if (i) the second analyte level for the second instance of the multiple instances of time is determined to be greater than the first analyte level threshold and (ii) the rate of change of the stream of second analyte levels at the second instance of the multiple instances of time is determined to be greater than or equal to the rising rate of change threshold.

B68. The apparatus of any one of embodiments B49-B67, wherein the processing circuitry is further configured to, subsequent to switching to use of the stream of second analyte levels for display: determine whether the first analyte level for a second instance of the multiple instances of time is less than a second analyte level threshold; if the first analyte level for the second instance of the multiple instances of time is determined to be less than the second analyte level threshold, determine whether the first analyte level for a third instance of the multiple instances of time is greater than the second analyte level threshold; and switch to use of the stream of first analyte levels for display if the first analyte level for the third instance of the multiple instances of time is determined to be greater than the second analyte level threshold.

B69. The apparatus of any one of embodiments B49-B67, wherein the processing circuitry is further configured to, subsequent to switching to use of the stream of second analyte levels for display: determine whether the second analyte level for a second instance of the multiple instances of time is less than a second analyte level threshold; if the second analyte level for the second instance of the multiple instances of time is determined to be less than the second analyte level threshold, determine whether the second analyte level for a third instance of the multiple instances of time is greater than the second analyte level threshold; and switch to use of the stream of first analyte levels for display if the second analyte level for the third instance of the multiple instances of time is determined to be greater than the second analyte level threshold.

B70. The apparatus of any one of embodiments B49-B67, wherein the processing circuitry is further configured to, subsequent to switching to use of the stream of second analyte levels for display: determine whether the first analyte level for a second instance of the multiple instances of time is less than a second analyte level threshold; if the first analyte level for the second instance of the multiple instances of time is determined to be less than the second analyte level threshold, (i) determine whether the first analyte level for a third instance of the multiple instances of time is greater than the second analyte level threshold and (ii) determine whether a rate of change of the stream of first analyte levels at the third instance of the multiple instances of time is greater than or equal to a rising rate of change threshold; and switch to use of the stream of first analyte levels for display if (i) the first analyte level for the third instance of the multiple instances of time is determined to be greater than the second analyte level threshold and (ii) the rate of change of the stream of first analyte levels at the third instance of the multiple instances of time is determined to be greater than or equal to the rising rate of change threshold.

B71. The apparatus of any one of embodiments B49-B67, wherein the processing circuitry is further configured to, subsequent to switching to use of the stream of second analyte levels for display: determine whether the second analyte level for a second instance of the multiple instances of time is less than a second analyte level threshold; if the second analyte level for the second instance of the multiple instances of time is determined to be less than the second analyte level threshold, (i) determine whether the second analyte level for a third instance of the multiple instances of time is greater than the second analyte level threshold and (ii) determine whether a rate of change of the stream of second analyte levels at the third instance of the multiple instances of time is greater than or equal to a rising rate of change threshold; and switch to use of the stream of first analyte levels for display if (i) the second analyte level for the third instance of the multiple instances of time is determined to be greater than the second analyte level threshold and (ii) the rate of change of the stream of second analyte levels at the third instance of the multiple instances of time is determined to be greater than or equal to the rising rate of change threshold.

B72. The apparatus of any one of embodiments B68-B71, wherein the second analyte level threshold is 70 mg/dL.

B73. The apparatus of any one of embodiments B68-B71, wherein the second analyte level threshold is 75 mg/dL.

B74. The apparatus of any one of embodiments B49-B73, wherein the first analyte level threshold is 100 mg/dL.

B75. The apparatus of any one of embodiments B49-B73, wherein the first analyte level threshold is 110 mg/dL.

B76. The apparatus of any one of embodiments B50-B75, wherein the maximum analyte level difference threshold is 40 mg/dL

B77. The apparatus of any of embodiments B40-B76, wherein the processing circuitry, in switching to use of the stream of second analyte levels for display, is configured to: use an average of the first and second analyte levels for one or more transitionary instances of the multiple instances of time for display and then using the second analyte levels for one or more instances of the multiple instances of time subsequent to the one or more transitionary instances.

B78. The apparatus of any one of embodiments B62-B77, wherein the processing circuitry, in switching to use of the stream of second analyte levels for display, is configured to:
use an average of the first and second analyte levels for one or more transitionary instances of the multiple instances of time for display and then using the first analyte levels for one or more instances of the multiple instances of time subsequent to the one or more transitionary instances.

What is claimed is:

1. A method comprising:
receiving sensor data for multiple instances of time, wherein the sensor data was conveyed by an analyte sensor;
using a first method to calculate a stream of first analyte levels for the multiple instances of time based on at least the sensor data for the multiple instances of time;
using a second method to calculate a stream of second analyte levels for the multiple instances of time based on at least the sensor data for the multiple instances of time;
using the stream of first analyte levels for display;
determining whether to switch from use of the stream of first analyte levels for display to use of the stream of second analyte levels for display, wherein determining whether to switch from use of the stream of first analyte levels for display to use of the stream of second analyte levels comprises comparing one or more of the first analyte levels to one or more of the second analyte levels;
continuing to use the stream of first analyte levels for display if determined to not switch from use of the stream of first analyte levels to use of the stream of second analyte levels; and
switching to use of the stream of second analyte levels for display if determined to switch from use of the stream of first analyte levels to use of the stream of second analyte levels.

2. The method of claim 1, further comprising:
receiving a first reference analyte value for a first reference instance of time; and
receiving a second reference analyte value for a second reference instance of time;
wherein determining whether to switch from use of the stream of first analyte levels for display to use of the stream of second analyte levels for display is performed if the second reference analyte value is received.

3. The method of claim 2, wherein comparing the one or more of the first analyte levels to the one or more of the second analyte levels includes:
determining an aggregate value of the first analyte levels that are for instances of the multiple instances of time that are between the first and second reference instances of time;
determining an aggregate value of the second analyte levels that are for instances of the multiple instances of time that are between the first and second reference instances of time; and
determining whether the aggregate value of the second analyte levels is lower than the aggregate value of the first analyte levels.

4. The method of claim 3, wherein determining whether to switch from use of the stream of first analyte levels for display to use of the stream of second analyte levels for display comprises determining to not switch from the use of the stream of first analyte levels for display to use of the stream of second analyte levels for display if the aggregate value of the second analyte levels is not determined to be lower than the aggregate value of the first analyte levels; and
determining whether to switch from use of the stream of first analyte levels for display to use of the stream of second analyte levels for display comprises determining to switch from the use of the stream of first analyte levels for display to use of the stream of second analyte levels for display if the aggregate value of the second analyte levels is determined to be lower than the aggregate value of the first analyte levels.

5. The method of claim 3, wherein the determined aggregate value of the first analyte levels that are for the instances of the multiple instances of time that are between the first and second reference instances of time is a first quartile of the first analyte levels that are for the instances of the multiple instances of time that are between the first and second reference instances of time, and the determined aggregate value of the second analyte levels that are for the instances of the multiple instances of time that are between the first and second reference instances of time is a first quartile of the second analyte levels that are for the instances of the multiple instances of time that are between the first and second reference instances of time.

6. The method of claim 2, further comprising:
receiving a third reference analyte value for a third reference instance of time;
determining a second aggregate value of the first analyte levels that are for instances of the multiple instances of time that are between the second and third reference instances of time;
determining a second aggregate value of the second analyte levels that are for instances of the multiple instances of time that are between the second and third reference instances of time;
determining whether the second aggregate value of the second analyte levels is lower than the second aggregate value of the first analyte levels;
using the stream of first analyte levels for display for one or more instances of the multiple instances of time that are subsequent to the third reference instances of time if the second aggregate value of the second analyte levels is not determined to be lower than the second aggregate value of the first analyte levels; and
using the stream of second analyte levels for display for the one or more instances of the multiple instances of time that are subsequent to the third reference instances of time if the second aggregate value of the second analyte levels is determined to be lower than the second aggregate value of the first analyte levels.

7. The method of claim 1, wherein determining whether to switch from use of the stream of first analyte levels for display to use of the stream of second analyte levels comprises (a) determining whether the first analyte level for a first instance of the multiple instances of time is less than a first analyte level threshold and/or (b) determining at the first instance of the multiple instances of time whether a predicted first analyte level is less than the first analyte level threshold;
wherein comparing the one or more of the first analyte levels to the one or more of the second analyte levels includes determining whether the second analyte level for the first instance of the multiple instances of time is lower than the first analyte level for the first instance of the multiple instances of time.

8. The method of claim 7, wherein comparing the one or more of the first analyte levels to the one or more of the second analyte levels further includes determining whether a difference between the first and second analyte levels for the first instance of the multiple instances of time is greater than a maximum analyte level difference threshold.

9. The method of claim 7, wherein determining whether to switch from use of the stream of first analyte levels for display to use of the stream of second analyte levels comprises determining whether the first analyte level for the first instance of the multiple instances of time is less than the first analyte level threshold.

10. The method of claim 7, wherein determining whether to switch from use of the stream of first analyte levels for display to use of the of the stream of second analyte levels for display comprises determining at the first instance of the multiple instances of time whether the predicted first analyte level is less than the first analyte level threshold.

11. The method of claim 7, wherein determining whether to switch from use of the stream of first analyte levels for display to use of the stream of second analyte levels for display comprises determining to not switch from the use of the stream of first analyte levels for display to use of the stream of second analyte levels for display if (a) the first analyte level for the first instance of time of the multiple instances of time is less than the second analyte level for the first instance of time and/or (b) the difference between the first and second analyte levels for the first instance of time is greater than the maximum analyte level difference threshold.

12. The method of claim 6, wherein determining whether to switch from use of the stream of first analyte levels for display to use of the stream of second analyte levels for display comprises determining to switch from the use of the stream of first analyte levels for display to use of the stream of second analyte levels for display if (i) the first analyte level for the first instance of time is greater than the second analyte level for the first instance of time and (ii) the difference between the first and second analyte levels for the first instance of time is less than the analyte difference threshold.

13. The method of claim 6, wherein determining whether to switch from use of the stream of first analyte levels for display to use of the stream of second analyte levels for display further comprises determining whether a rate of change of the stream of first analyte levels at the first instance of the multiple instances of time is less than or equal to a falling rate of change threshold.

14. The method of claim 13, wherein determining whether to switch from use of the stream of first analyte levels for display to use of the stream of second analyte levels for display comprises determining to not switch from the use of the stream of first analyte levels for display to use of the stream of second analyte levels for display if the rate of change of the stream of first analyte levels at the first instance of the multiple instances of time is determined to be not less than or equal to the falling rate of change threshold.

15. The method of claim 6, wherein comparing the one or more of the first analyte levels to the one or more of the second analyte levels further includes determining whether the difference between the first and second analyte levels for the first instance of the multiple instances of time is less than a minimum analyte level difference threshold.

16. The method of claim 15, wherein determining whether to switch from use of the stream of first analyte levels for display to use of the stream of second analyte levels for display comprises determining to not switch from the use of the stream of first analyte levels for display to use of the stream of second analyte levels for display if the difference between the first and second analyte levels for the first instance of the multiple instances of time is determined to be less than the minimum analyte level difference threshold.

17. The method of claim 6, wherein comparing the one or more of the first analyte levels to the one or more of the second analyte levels further includes comparing a minimum cost of the stream of first analyte levels at a last reference analyte value to a minimum cost of the stream of second analyte levels at the last reference analyte value.

18. The method of claim 17, wherein determining whether to switch from use of the stream of first analyte levels for display to use of the stream of second analyte levels for display comprises determining to not switch from the use of the stream of first analyte levels for display to use of the stream of second analyte levels for display if the minimum cost of the stream of second analyte levels at the last reference analyte value is not less than a sum of the minimum cost of the stream of first analyte levels at the last reference analyte value and a minimum cost difference threshold.

19. The method of claim 6, wherein determining whether to switch from use of the stream of first analyte levels for display to use of the stream of second analyte levels for display further comprises determining whether a minimum cost of the stream of second analyte levels at the last reference analyte value is less than a maximum cost threshold.

20. The method of claim 19, wherein determining whether to switch from use of the stream of first analyte levels for display to use of the stream of second analyte levels for display comprises determining to not switch from the use of the stream of first analyte levels for display to use of the stream of second analyte levels for display if the minimum cost of the stream of second analyte levels at the last reference analyte value is determined to be not less than the maximum cost threshold.

21. The method of claim 6, further comprising, subsequent to switching to use of the stream of second analyte levels for display:
determining whether the first analyte level for a second instance of the multiple instances of time is greater than the first analyte level threshold; and
switching to use of the stream of first analyte levels for display if the first analyte level for the second instance of the multiple instances of time is determined to be greater than the first analyte level threshold.

22. The method of claim 6, further comprising, subsequent to switching to use of the stream of second analyte levels for display:
determining whether the second analyte level for a second instance of the multiple instances of time is greater than the first analyte level threshold; and
switching to use of the stream of first analyte levels for display if the second analyte level for the second instance of the multiple instances of time is determined to be greater than the first analyte level threshold.

23. The method of claim 6, further comprising, subsequent to switching to use of the stream of second analyte levels for display:
determining whether the first analyte level for a second instance of the multiple instances of time is greater than the first analyte level threshold;
determining whether a rate of change of the stream of first analyte levels at the second instance of the multiple instances of time is greater than or equal to a rising rate of change threshold; and
switching to use of the stream of first analyte levels for display if (i) the first analyte level for the second instance of the multiple instances of time is determined to be greater than the first analyte level threshold and (ii) the rate of change of the stream of first analyte levels at the second instance of the multiple instances of time is determined to be greater than or equal to the rising rate of change threshold.

24. The method of claim 6, further comprising, subsequent to switching to use of the stream of second analyte levels for display:
determining whether the second analyte level for a second instance of the multiple instances of time is greater than the first analyte level threshold;
determining whether a rate of change of the stream of second analyte levels at the second instance of the multiple instances of time is greater than or equal to a rising rate of change threshold; and
switching to use of the stream of first analyte levels for display if (i) the second analyte level for the second instance of the multiple instances of time is determined to be greater than the first analyte level threshold and (ii) the rate of change of the stream of second analyte levels at the second instance of the multiple instances of time is determined to be greater than or equal to the rising rate of change threshold.

25. The method of claim 6, further comprising, subsequent to switching to use of the stream of second analyte levels for display:
determining whether the first analyte level for a second instance of the multiple instances of time is less than a second analyte level threshold;
if the first analyte level for the second instance of the multiple instances of time is determined to be less than the second analyte level threshold, determining whether the first analyte level for a third instance of the multiple instances of time is greater than the second analyte level threshold; and
switching to use of the stream of first analyte levels for display if the first analyte level for the third instance of the multiple instances of time is determined to be greater than the second analyte level threshold.

26. The method of claim 6, further comprising, subsequent to switching to use of the stream of second analyte levels for display:
determining whether the second analyte level for a second instance of the multiple instances of time is less than a second analyte level threshold;
if the second analyte level for the second instance of the multiple instances of time is determined to be less than the second analyte level threshold, determining whether the second analyte level for a third instance of the multiple instances of time is greater than the second analyte level threshold; and
switching to use of the stream of first analyte levels for display if the second analyte level for the third instance of the multiple instances of time is determined to be greater than the second analyte level threshold.

27. The method of claim 6, further comprising, subsequent to switching to use of the stream of second analyte levels for display:
determining whether the first analyte level for a second instance of the multiple instances of time is less than a second analyte level threshold;
if the first analyte level for the second instance of the multiple instances of time is determined to be less than the second analyte level threshold, (i) determining whether the first analyte level for a third instance of the multiple instances of time is greater than the second analyte level threshold and (ii) determining whether a rate of change of the stream of first analyte levels at the third instance of the multiple instances of time is greater than or equal to a rising rate of change threshold; and
switching to use of the stream of first analyte levels for display if (i) the first analyte level for the third instance of the multiple instances of time is determined to be greater than the second analyte level threshold and (ii) the rate of change of the stream of first analyte levels at the third instance of the multiple instances of time is determined to be greater than or equal to the rising rate of change threshold.

28. The method of claim 6, further comprising, subsequent to switching to use of the stream of second analyte levels for display:
determining whether the second analyte level for a second instance of the multiple instances of time is less than a second analyte level threshold;
if the second analyte level for the second instance of the multiple instances of time is determined to be less than the second analyte level threshold, (i) determining whether the second analyte level for a third instance of the multiple instances of time is greater than the second analyte level threshold and (ii) determining whether a rate of change of the stream of second analyte levels at the third instance of the multiple instances of time is greater than or equal to a rising rate of change threshold; and
switching to use of the stream of first analyte levels for display if (i) the second analyte level for the third instance of the multiple instances of time is determined to be greater than the second analyte level threshold and (ii) the rate of change of the stream of second analyte levels at the third instance of the multiple instances of time is determined to be greater than or equal to the rising rate of change threshold.

29. The method of claim 1, wherein switching to use of the stream of second analyte levels for display comprises using an average of the first and second analyte levels for one or more transitionary instances of the multiple instances of time for display and then using the second analyte levels for one or more instances of the multiple instances of time subsequent to the one or more transitionary instances.

30. An apparatus comprising:
an antenna configured to receive sensor data for multiple instances of time, wherein the sensor data is conveyed by an analyte sensor; and
processing circuitry configured to:
use a first method to calculate a stream of first analyte levels for the multiple instances of time based on at least the sensor data for the multiple instances of time;
use a second method to calculate a stream of second analyte levels for the multiple instances of time based on at least the sensor data for the multiple instances of time;
use the stream of first analyte levels for display;
determine whether to switch from use of the stream of first analyte levels for display to use of the stream of second analyte levels for display, wherein determining whether to switch from use of the stream of first analyte levels for display to use of the stream of second analyte levels comprises comparing one or more of the first analyte levels to one or more of the second analyte levels;
continue to use the stream of first analyte levels for display if determined to not switch from use of the stream of first analyte levels to use of the stream of second analyte levels; and
switch to use of the stream of second analyte levels for display if determined to switch from use of the stream of first analyte levels to use of the stream of second analyte levels.
